SEQUENCE LISTING (24 sequences)

SEQ ID NO: 1 - protein, 441 aa, Homo sapiens
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 2 - protein, 412 aa, Homo sapiens
MAEPRQEFEVMEDHAGTYGLGDRKDQGGYTMHQDQEGDTDAGLKESPLQTPTEDGSEEPGSETSDAKSTPTAEAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 3 - protein, 383 aa, Homo sapiens
MAEPRQEFEVMEDHAGTYGLGDRKDQGGYTMHQDQEGDTDAGLKAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 4 - protein, 410 aa, Homo sapiens
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 5 - protein, 381 aa, Homo sapiens
MAEPRQEFEVMEDHAGTYGLGDRKDQGGYTMHQDQEGDTDAGLKESPLQTPTEDGSEEPGSETSDAKSTPTAEAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 6 - protein, 352 aa, Homo sapiens
MAEPRQEFEVMEDHAGTYGLGDRKDQGGYTMHQDQEGDTDAGLKAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 7 - protein, 129 aa, Homo sapiens
QTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIE

SEQ ID NO: 8 - protein, 441 aa, Homo sapiens
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVLGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 9 - protein, 449 aa, Artificial Sequence
  HUMAN WILD TYPE TAU-FLAG
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGLDYKDDDDK

SEQ ID NO: 10 - protein, 449 aa, Artificial Sequence
  HUMAN P301L MUTANT TAU-FLAG
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVLGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGLDYKDDDDK

SEQ ID NO: 11 - protein, 912 aa, Artificial Sequence
  TAU TANDEM REPEAT POLYPEPTIDE- TAU-LINKER- TAU-FLAG
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGLGSGATNFSLLKQAGDVEENAVPMAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGLDYKDDDDK

SEQ ID NO: 12 - protein, 890 aa, Artificial Sequence
  TAU TANDEM REPEAT POLYPEPTIDE-TAU-TAU-FLAG
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGLMAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGLDYKDDDDK

SEQ ID NO: 13 - protein, 895 aa, Artificial Sequence
  TAU TANDEM REPEAT POLYPEPTIDE-TAU-QQQQS- TAU-FLAG
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGLQQQQSMAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGLDYKDDDDK

SEQ ID NO: 14 - protein, 915 aa, Artificial Sequence
  TAU TANDEM REPEAT POLYPEPTIDE-TAU-(QQQQS)5X- TAU-FLAG
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGLQQQQSQQQQSQQQQSQQQQSQQQQSMAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGLDYKDDDDK

SEQ ID NO: 15 - protein, 600 aa, Artificial Sequence
  TAU TANDEM REPEAT POLYPEPTIDE-TAU-P2AM-K18-FLAG
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGLGSGATNFSLLKQAGDVEENAVPQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIEDYKDDDDK

SEQ ID NO: 16 - protein, 357 aa, Artificial Sequence
  dNTERM TAU(244-441)-P2aM-K18-FLAG
QTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGLGSGATNFSLLKQAGDVEENAVPQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIEDYKDDDDK

SEQ ID NO: 17 - protein, 22 aa, Artificial Sequence
  LINKER
GSGATNFSLLKQAGDVEENAVP

SEQ ID NO: 18 - protein, 5 aa, Artificial Sequence
  LINKER
QQQQS

SEQ ID NO: 19 - protein, 25 aa, Artificial Sequence
  LINKER
QQQQSQQQQSQQQQSQQQQSQQQQS

SEQ ID NO: 20 - protein, 8 aa, Artificial Sequence
  FLAG EPITOPE
DYKDDDDK

SEQ ID NO: 21 - protein, 6 aa, Artificial Sequence
  HIS EPITOPE TAG
HHHHHH

SEQ ID NO: 22 - protein, 9 aa, Artificial Sequence
  HA EPITOPE TAG
YPYDVPDYA

SEQ ID NO: 23 - protein, 10 aa, Artificial Sequence
  MYC EPITOPE TAG
EQKLISEEDL

SEQ ID NO: 24 - protein, 14 aa, Artificial Sequence
  V5 EPITOPE TAG
GKPIPNPLLGLDST

(19) United States
(12) Patent Application Publication
(10) Pub. No.: US 2018/0164288 A1
(43) Pub. Date: Jun. 14, 2018

(54) CELLULAR EXPRESSION MODEL OF TAU AGGREGATION

(71) Applicant: Merck Sharp & Dohme Corp., Rahway, NJ (US)

(72) Inventors: Joel Schachter, Lansdale, PA (US); John Majercak, Wayne, PA (US); Mali Liu Cosden, Fort Washington, PA (US)

(73) Assignee: Merck Sharp & Dohme Corp., Rahway, NJ (US)

(21) Appl. No.: 15/378,443

(22) Filed: Dec. 14, 2016

(51) Int. Cl. G01N 33/50; C07K 14/47

(52) U.S. Cl. CPC G01N 33/5008; C07K 2319/43; G01N 2333/47; C07K 14/47

(57) ABSTRACT

The invention relates to novel methods and compositions for detecting tau hyperphosphorylation and aggregation.

### FIELD OF THE INVENTION

The invention relates to novel methods and compositions for detecting tau hyperphosphorylation and aggregation.

### BACKGROUND OF THE INVENTION

Neurofibrillary lesions composed of hyperphosphorylated aggregates of the microtubule-associated protein tau are classic histological features found in brains from Alzheimer's patients. Tau pathologies are also detected in a number of other neurodegenerative diseases such as Pick's disease, Progressive Supranuclear Palsy, Corticobasilar Degeneration, and Frontotemporal Dementia with Parkinsonism, that are collectively referred to as Tauopathies. In the case of Alzheimer's disease, it is well established that the magnitude and localization of tau pathology correlates with clinical disease progression and neurodegeneration (1). The question of whether tau pathology is a primary driver or a secondary consequence of neurodegenerative processes has not been fully answered, but the clinical findings that aggregation-promoting mutations in the tau coding sequence underlie an inheritable form of Frontotemporal Dementia (2-6) is strongly suggestive that tau aggregation precedes and promotes neurodegeneration. This suggestion is supported by the presence of tau pathology and neurodegeneration in transgenic mouse models that express aggregation-prone FTD mutant forms of tau (7-9) and by studies in which microinjection of small soluble tau aggregates (10, 11) or sonicated tau fibrils (12) into rodent brain induced tauopathy and neurodegeneration.

Despite the strong correlation of neurofibrillary pathology to neurodegeneration and disease progression in Alzheimer's disease and other Tauopathies, recent studies in transgenic mouse models of tauopathy and in human post-mortem brains suggest that the large tau assemblies comprising NFT's may not specifically be the causal agents for tau-related neurodegeneration. The Tg4510 mouse model of Tauopathy inducibly over-expresses a P301L mutation of human tau that is causally associated with a subset of familial FTD (8). Induction of mutant tau expression in this mouse produces NFT pathology and neurodegeneration. Using this model, Santacruz demonstrated that cessation of mutant tau expression could halt neurodegenerative progression in Tg4510, despite the persistence of neurofibrillary pathology (13). This initial dissociation of tau neurofibrillary pathology from neurodegeneration was followed by several other observations, consistent with a distinction between fibrillar tau pathology and tau-induced neurodegeneration(14, 15). In the h-tau mouse model that expresses wild type human tau, neurodegeneration, synaptic loss, and behavioral impairment occur in the absence of somatic NFTs (16, 17). In another mutant tau mouse expressing the P301S mutation, hippocampal synapse loss and microgliosis were seen to precede NFT formation (9). Similarly, stereologic studies of Braak-staged human AD brains show that regional neuronal loss often precedes localized NFT formation (18, 19). These findings suggested that other factors, such as non-fibrillar species of tau aggregates, may be present that are responsible for neurodegenerative activities.

The role of small soluble oligomeric protein aggregates has recently received considerable interest in several neurodegenerative diseases. Oligomers of Abeta, a-synuclein, and prion have all been linked to neurotoxicity or cognitive dysfunction (20-24). In the case of tau, small soluble tau oligomers have been detected in brains of Tg4510 mice and in the brains of Alzheimer's patients (25-27). Injection of tau oligomers, but not tau monomers or fibrils, into the brains of wild-type mice has produced neurodegeneration and impaired memory function (10). Based upon these types of studies, therapeutic efforts in the field are gradually shifting away from a sole focus on neurofibrillary pathology and more toward a concentration on preventing or reversing accumulation of smaller tau oligomers.

Early efforts to model tau pathology relied heavily on in vitro tau aggregation assays in which purified or recombinant protein was incubated at relatively high concentration in the presence of anionic reagents. Under these conditions tau forms filamentous structures that strongly resemble the paired helical filaments that comprise NFT's. Generation of tau filaments in vitro is thought to involve an initial nucleation event, followed by a progressive elongation reaction in which monomers are sequentially added to the nucleated species (33, 34). Nucleation is thought to be the rate-limiting step in this process, whereas elongation is energetically favored. The energy barrier to tau aggregate nucleation in vitro can be reduced by the presence of anions, such as arachidonic acid, heparin, or RNA (35, 36). Photoactivated cross-linking studies during the initial stages of in vitro tau aggregation indicate that the first step in the nucleation process is the formation of tau dimers (26). These dimers subsequently form insoluble tau filaments through sequential elongation. Based upon physicochemical and immunogenicity analyses, it has been suggested that tau filament formation involves an antiparallel stacking of tau monomers (26, 37).

While In Vitro aggregation of tau into PHF's was demonstrated decades ago (28-30) and has been widely evaluated, there have been only a small number of reports describing tau aggregation in cellular systems. Cellular transfection with cDNAs encoding full length tau, or full length tau containing FTD mutations, typically results in stable expression of monomeric tau. Tau aggregation and tau hyperphosphorylation are difficult to detect in these cellular systems. Recently, cellular models expressing FTD mutant forms of the microtubule-binding fragment of tau, termed either K18 (31) or tau C-terminal repeat domain (RD) (32), have been reported to display some degree of spontaneous aggregation when expressed in mammalian cells. These models have utilized a small fragment of tau that is devoid of most of the AD-relevant phosphorylation sites and they require the use of FTD-related mutations to promote the aggregation. Thus, there is still a need in the art for cellular model systems that demonstrate hyperphosphorylation and aggregation of full length wild type tau.

Here we report a cellular model system in which full-length, wild-type human tau is expressed as a single chain, tandem repeat sequence to promote intramolecular folding and mimic the antiparallel stacking conformation of naturally occurring tau dimers. Inducible expression of this construct in mammalian cells results in the rapid generation of hyperphosphorylated tau and is followed by a slower accumulation of multi-molecular tau aggregates. These cellular tau aggregates bind the beta sheet sensitive reagents, thioflavin-S and primulin. High speed sedimentation of cell extracts reveals proteolytic generation of hyperphosphorylated tau monomers and fragments that concentrate in the detergent-insoluble high speed pellet. This new cellular model of tau hyperphosphorylation and aggregation should be useful in elucidating basic cellular mechanisms associated with tau aggregation and pathology. Furthermore this model provides a platform assay for drug discovery efforts targeting tauopathies.

### SUMMARY OF THE INVENTION

The invention comprises a tau tandem repeat polypeptide comprising a structure selected from the group consisting of:
X₁-L-X₂-T,
X₂-L-X₁-T,
X₁-X₂-T, or
X₂-X₁-T
   wherein: X₁=Tau monomer, L=linker, X₂=Tau monomer or a fragment thereof comprising the K18 fragment; and T=detectable tag.

In one embodiment, X₁ could be selected from the group consisting of: SEQ ID NO: 1, SEQ ID NO: 2; SEQ ID NO: 3; SEQ ID NO: 4, SEQ ID NO: 5, and SEQ ID NO: 6.

In one embodiment, X₂ could be selected from the group consisting of: SEQ ID NO: 1, SEQ ID NO: 2; SEQ ID NO: 3; SEQ ID NO: 4, SEQ ID NO: 5, and SEQ ID NO: 6 or a fragment of any one of SEQ ID NOs: 1-6 containing the K18 repeat domain. In one embodiment, X₂=SEQ ID NO:7.

In any of the embodiments that require a linker, the linker could be any amino acid sequence comprising from 1 to 450 amino acids, as long as the linker allows for the transcription of the tau tandem repeat as a single protein. In one embodiment, the linker is selected from a group consisting of: SEQ ID NO:17 (GSGATNFSLLKQAGDVEENAVP, “mutP2A” or “P2aM”), SEQ ID NO:18 (QQQQS) or SEQ ID NO:19 (5X-QQQQS). In one embodiment, the linker is SEQ ID NO:17 (GSGATNFSLLKQAGDVEENAVP)

In one embodiment, the detectable tag could be any tag or epitope detectable by an antibody. In one embodiment, the detectable is selected from the group consisting of: FLAG (SEQ ID NO:20), HA (SEQ ID NO22), myc (SEQ ID NO:23), 6-His (SEQ ID NO21), V5 (SEQ ID NO:24), GST (Glutathione-S-Transferase), GFP (Green Fluorescent Protein). In one embodiment, the detectable tag is FLAG.

In one embodiment, X₁=SEQ ID NO:1, L=SEQ ID NO:17, X₂=SEQ ID NO:1 or SEQ ID NO:7; and T=FLAG.

In one embodiment, the tau tandem repeat polypeptide comprises or consists of any one of the amino acid sequences of SEQ ID NOs:11-16. In one embodiment, the tau tandem repeat polypeptide comprises or consists of the amino acid sequence of SEQ ID NO:11. In one embodiment, the tau tandem repeat polypeptide comprises or consists of the amino acid sequence of SEQ ID NO:12. In one embodiment, the tau tandem repeat polypeptide comprises or consists of the amino acid sequence of SEQ ID NO:13. In one embodiment, the tau tandem repeat polypeptide comprises or consists of the amino acid sequence of SEQ ID NO:14. In one embodiment, the tau tandem repeat polypeptide comprises or consists of the amino acid sequence of SEQ ID NO:15. In one embodiment, the tau tandem repeat polypeptide comprises or consists of the amino acid sequence of SEQ ID NO:16.

The invention also comprises a DNA construct encoding a tau tandem repeat polypeptide comprising a structure selected from the group consisting of: X₁-L-X₂-T, X₂-L-X₁-T, X₁-X₂-T, or X₂-X₁-T; wherein: X₁=Tau monomer, L=linker, X₂=Tau monomer or a fragment thereof comprising the K18 fragment; and T=detectable tag. In one embodiment, X₁=SEQ ID NO:1, L=SEQ ID NO:17, X₂=SEQ ID NO:1 or SEQ ID NO:7; and T=FLAG.

The invention also comprises a host cell expressing a DNA construct encoding a tau tandem repeat polypeptide comprising a structure selected from the group consisting of: X₁-L-X₂-T, X₂-L-X₁-T, X₁-X₂-T, or X₂-X₁-T; wherein: X₁=Tau monomer, L=linker, X₂=Tau monomer or a fragment thereof comprising the K18 fragment; and T=detectable tag. In one embodiment, X₁=SEQ ID NO:1, L=SEQ ID NO:17, X₂=SEQ ID NO:1 or SEQ ID NO:7; and T=FLAG.

The invention also comprises a non-human transgenic mammal expressing a tau tandem repeat polypeptide comprising a structure selected from the group consisting of: X₁-L-X₂-T, X₂-L-X₁-T, X₁-X₂-T, or X₂-X₁-T; wherein: X₁=Tau monomer, L=linker, X₂=Tau monomer or a fragment thereof comprising the K18 fragment; and T=detectable tag. In one embodiment, X₁=SEQ ID NO:1, L=SEQ ID NO:17, X₂=SEQ ID NO:1 or SEQ ID NO:7; and T=FLAG. In one embodiment, the non-human transgenic mammal is used to screen for inhibitors of tau aggregation. In one embodiment, the non-human transgenic mammal is used to screen for agents that can be used to treat a tauopathy. In one embodiment, the non-human transgenic mammal is a mouse.

The invention also comprises a method for screening for inhibitors of tau aggregation comprising: (i) contacting a test compound with a cell expressing a tau tandem repeat for a time sufficient to allow the formation of tau tandem repeat oligomers, wherein the tau tandem repeat comprises a structure selected from the group consisting of: X₁-L-X₂-T, X₂-L-X₁-T, X₁-X₂-T, or X₂-X₁-T; wherein: X₁=Tau monomer, L=linker, X₂=Tau monomer or a fragment thereof comprising the K18 fragment; and T=detectable tag, (ii) measuring the presence of tau tandem repeat oligomers using an assay that is able to differentiate between tau tandem repeats monomers and tau tandem repeat oligomers; wherein a decrease in the presence of tau tandem repeat oligomers relative to control indicates that the compound is an inhibitor of tau aggregation. In one embodiment, X₁=SEQ ID NO:1, L=SEQ ID NO:17, X₂=SEQ ID NO:1 or SEQ ID NO:7; and T=FLAG. In one embodiment, the method further comprises adding an agent that facilitates the formation of tau oligomers (e.g., K18 repeat domain, heparin, oligomer seeds) between steps (i) and (ii). In one embodiment, the measuring step (step (ii)) uses a bead based immunoassay comprising an acceptor bead linked to a first antibody and an donor bead linked to a second antibody (for example, an AlphaLISA® assay from Perkin Elmer). In one embodiment, the acceptor bead and the first antibody are linked by the interaction of streptavidin and biotin. In one embodiment, the acceptor bead is coated with streptavidin and the first antibody is linked to biotin, and the donor bead and the second antibody are linked by cyanoborohydride activation. In one embodiment, the acceptor bead and the first antibody are linked by cyanoboronhydride activation, the donor bead is coated with streptavidin and the and the second antibody is linked to biotin. In one embodiment, the first and the second antibody specifically bind to the detectable tag (T), which can be FLAG. Compounds identified by this method can be used to treat tauopathies.

The invention also comprises a method for screening for enhancers of tau oligomer degradation comprising: (i) contacting a test compound with a cell expressing a tau tandem repeat for a time sufficient to allow the formation of tau tandem repeat oligomers, wherein the tau tandem repeat comprises a structure selected from the group consisting of: X₁-L-X₂-T, X₂-L-X₁-T, X₁-X₂-T, or X₂-X₁-T; wherein: X₁=Tau monomer, L=linker, X₂=Tau monomer or a fragment thereof comprising the K18 fragment; and T=detectable tag, (ii) measuring the presence of tau tandem repeat oligomers using an assay that is able to differentiate between tau tandem repeats monomers and tau tandem repeat oligomers; wherein a decrease in the presence of tau oligomers relative to control indicates that the compound is an enhancer of tau degradation. In one embodiment, X₁=SEQ ID NO:1, L=SEQ ID NO:17, X₂=SEQ ID NO:1 or SEQ ID NO:7; and T=FLAG. In one embodiment, the cell expressing the tau tandem repeat needs to be induced to express the tau tandem repeat prior to step (i). In one embodiment, the method further comprises adding an agent that facilitates the formation of tau oligomers (e.g. K18 repeat domain, heparin, oligomer seeds) between steps (i) and (ii). In one embodiment, the measuring step (step (ii)) uses a bead based immunoassay comprising an acceptor bead linked to a first antibody and an donor bead linked to a second antibody (for example, an AlphaLISA® assay from Perkin Elmer). In one embodiment, the acceptor bead and the first antibody are linked by the interaction of streptavidin and biotin. In one embodiment, the acceptor bead is coated with streptavidin and the first antibody is linked to biotin, and the donor bead and the second antibody are linked by cyanoborohydride activation. In one embodiment, the acceptor bead and the first antibody are linked by cyanoboronhydride activation, the donor bead is coated with streptavidin and the second antibody is linked to biotin. In one embodiment, the first and the second antibody specifically bind to the detectable tag (T), which can be FLAG. Compounds identified by this method can be used to treat tauopathies.

### BRIEF DESCRIPTION OF THE DRAWINGS

FIG. 1. Validation of Immunoassays used for detection of tau oligomers. (A) Synthetic tau oligomers were produced by aggregation of recombinant human tau monomers for different times in the presence of heparin and tau tandem repeat polypeptide. Tau oligomers and tau filaments were then detected at different dilutions with a bead-based sandwich immunoassay utilizing the HT7 tau antibody on both sides of the sandwich. (B) A second sandwich immunoassay, utilizing FLAG antibodies on both sides of the sandwich was validated with a peptide consisting of three consecutive copies of FLAG peptide (3×FLAG) and its specificity was demonstrated by the use of a peptide consisting of a single copy of FLAG peptide (1×FLAG).

FIG. 2. Demonstration of aggregate formation by Tandem Repeat Tau. Map showing cDNA constructs for wild type tau and for Tandem Repeat Tau both containing a FLAG epitope tag.

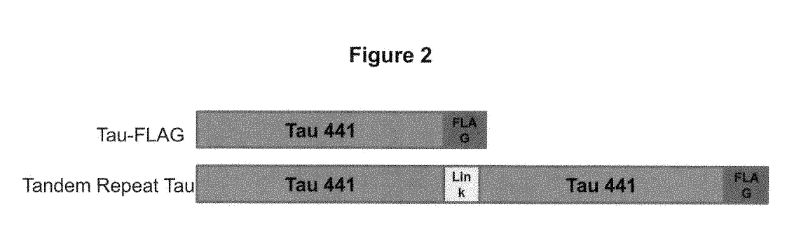

FIG. 3. Size exclusion FPLC of tau oligomers. Cellular lysates from stable, inducible HEK293 cell lines expressing either wild type human tau monomers or expressing tau tandem repeat (tau-mutP2A-tau) were fractionated using Superose 6 FPLC and assessed for tau oligomers and for total tau. The figure shows representative column runs from both cell lines combined into a single graph. Column fractions of extracts from wild type tau monomer cells were diluted to be within the linear range of the assay and then assayed for total tau (black filled circles). Fractions from a separate column run of cells expressing tandem repeat tau were assayed with two different immunoassays. Total tau was detected using the same immunoassay as was used for tau monomers at a similar dilution factor. Tau aggregates were detected with the FLAG/FLAG immunoassay. No tau aggregates were detected in the fractions containing tau monomers. Aggregates were detected in the high molecular weight fractions containing tau tandem repeat.

FIG. 4. Time course for induction of tau expression, hyperphosphorylation, and aggregation. A stable cell line with inducible expression of tandem repeat tau was seeded in multi-well plates and different wells were induced at various time points over the course of 6 days. All wells were harvested and assayed on day 6. The total tau immunoassay used HT7 and BT2 antibodies. The phosphotau assay used HT7 total tau capture and AT8 phosphorylation-specific detection antibody. FLAG-tagged tau aggregates were detected with the FLAG/FLAG immunoassay.

FIG. 5. Evaluation of tau phosphorylation at multiple epitopes. Two stable cell lines expressing either wild type tau monomer or tandem repeat tau were induced for four days and then cell lysates were examined by a series of phospho-specific antibodies and total tau antibodies. The lysates were diluted to generate similar immunoreactivity in several total tau assays consisting of HT7 or DA9 capture antibodies and BT2, DA31, or DAKO detection antibodies. Total tau data is shown from the HT7/BT2 assay. Using these relative dilutions, the two lysates were examined with a series of phosphotau-specific detection antibodies in conjunction with HT7 capture. All assays were individually optimized to ensure the lysates were measured in the linear range of the assay.

FIG. 6. Comparison of cDNA constructs. This figure shows construct maps for the coding regions of various chimeric tau constructs that were examined for promoting aggregation. As shown in the Figure, various linker sequences were introduced between the two copies of full length human tau. Further, the minimal tau sequence requirements for aggregation were examined using truncated constructs of tau. Additionally, the role for the covalent coupling of the two tau sequences was examined by mutating the 20^{th} and 21^{st} amino acids in the linker region (AVP converted to PGP) to generate a competent picornavirus P2A linker region. This resulted in generation of separate tau and K18-FLAG proteins, as shown by Western blot (data not shown). A second P2A construct encoding Tau and mutant K18-FLAG was also generated in which the K18 sequence was modified by deletion of K280 and mutation of V337M.

FIG. 7. Measurement of Tau Oligomers using Different cDNA Constructs. This figure shows the detection of Tau Oligomers in HEK293 cells transiently transfected with various tau expression plasmids. Tau expression plasmids were transiently transfected in HEK293 cells for 72 hours and then cell lysates were assayed for the presence of tau aggregates by FLAG/FLAG AlphaLisa® assay described herein.

FIG. 8. Incorporation of wild type tau monomers into aggregates formed by tandem repeat tau. Stable cells with inducible expression of wild type human tau monomer were transiently transfected with FLAG-tagged Tandem Repeat Tau that was modified with mouse tau sequence at both of the HT7 binding sites. This modified construct did not display any binding to HT7 antibody. Following transfection of Tandem Repeat Tau the FLAG/FLAG assay shows Tandem Repeat Tau aggregation, in the absence and in the presence of tau monomer induction. A second immunoassay consisting of HT7 capture of tau monomers and FLAG detection of Tandem Repeat Tau demonstrates incorporation of monomer into the Tandem Repeat Tau aggregates during co-expression of both proteins.

### DETAILED DESCRIPTION OF THE INVENTION

As used herein a “tau tandem repeat polypeptide” or “tandem repeat tau” or “tau tandem repeat monomer” refers to a polypeptide comprising the structure:
X₁-L-X₂-T,
X₂-L-X₁-T,
X₁-X₂-T, or
X₂-X₁-T
   wherein: X₁=Tau monomer, L=linker, X₂=Tau monomer or a fragment thereof comprising the K18 fragment; and T=detectable tag.

As used herein, the term “Tau monomer” refers to any one of the isoforms of tau from any species. The term “human Tau” or “human Tau monomer” refers to all known isoforms of human tau. There are six major isoforms of tau expressed in the adult human brain, all of which are derived from a single gene by alternative splicing. From a structural stand-point, tau is characterized by the presence of a MT-binding domain, which is composed of repeats of highly conserved tubulin-binding motif and which comprises the carboxy-terminal (C-terminal) half of the protein, followed by a basic proline-rich region and an acidic amino-terminal region, which is normally referred to as the projection domain. The six tau isoforms differe from each other in the number of tubulin-binding repeats (either three or four, hence the isoforms are normally referred to as 3R and 4R tau isoforms, respectively) and in the presence or absence of either one or two 29 amino acid long inserts at the N-terminal portion of the protein. These six isoforms are known as: 4R/2N tau (NP_005901; SEQ ID NO:1), 4R/1N (SEQ ID NO:2), 4R/0N(SEQ ID NO:3), 3R/2N (SEQ ID NO:4), 3R/1N(SEQ ID NO:5), 3R/0N (SEQ ID NO: 6). For a detailed description of the known isoforms of human tau, see, e.g., Ballatore et al., Nature Reviews, Volume 8, pages 663-672 (2007).

As used herein the “K18 fragment of tau” or “K18 tau” or “K18 fragment” refers to a fragment of any of the tau isoforms comprising aa 244-372 of SEQ ID NO:1). This fragment comprises the sequence shown in SEQ ID NO:7.

As used herein a “tau tandem repeat oligomer” refers to tau aggregates which comprise at least two tau tandem repeat polypeptides. In some embodiments, the oligomers will comprise between two to twenty (2) tau tandem repeat polypeptides.

As used herein the term “linker” refers to any amino acid linker comprising 1 to 451 amino acids in lengths, as long as the linker allows for the translation of the tau tandem repeat structure as a single protein.

As used herein “tauophathy” refers to tau-related disorders or conditions, e.g., Alzheimer's Disease, Progressive Supranuclear Palsy (PSP), Corticobasal Degeneration (CBD), Pick's Disease, Frontotemporal dementia and Parkinsonism associated with chromosome 17 (FTDP-17), Parkinson's disease, stroke, traumatic brain injury, mild cognitive impairment and the like”.

### Detectable Tags

Any tag or epitope that can be detected by an antibody could be used in the claimed tau tandem repeat polypeptides. In one embodiment, the polypeptide can comprise the FLAG epitope (DYKDDDDK; SEQ ID NO:20), and could be detected by an FLAG epitope tag antibody. In another embodiment, the tau tandem polypeptide can comprise a His epitope tag (6x-His; SEQ ID NO:21), and could be detected by a His epitope tag antibody. In another embodiment, the tau tandem polypeptide can comprise a HA Epitope Tag (YPYDVPDYA; SEQ ID NO:22), and could be detected by a HA Epitope Tag antibody. In another embodiment, the tau tandem polypeptide can comprise a Myc Epitope Tag (EQKLISEEDL; SEQ ID NO:23), and could be detected by a Myc Epitope Tag antibody. In another embodiment, the tau tandem polypeptide can comprise a GST Tag (Glutathione-S-Transferase), and could be detected by GST Tag Antibody. In another embodiment, the tau tandem polypeptide can comprise a GFP Tag (Green Fluorescent Protein) could be detected by GFP Tag Antibody. In another embodiment, the tau tandem polypeptide can comprise a V5 Epitope Tag (GKPIPNPLLGLDST; SEQ ID NO:24) could be detected by V5 Epitope Tag Antibody. Antibodies against these tags are well known and available in the art.

### Methods of Detecting Tau Oligomers

The claimed screening assays could use any method of detecting the presence of tau tandem repeat oligomers as long as the assay is able to differentiate between tau tandem repeats monomers and tau tandem repeat oligomers. One such assay exemplified herein uses AlphaLISA® which is a bead-based assay technology used to study biomolecular interactions in a microplate format. The acronym “Alpha” stands for amplified luminescent proximity homogeneous assay. As the name implies, some of the key features of these technologies are that they are non-radioactive, homogeneous proximity assays. Binding of molecules captured on the beads leads to an energy transfer from one bead to the other, ultimately producing a luminescent/fluorescent signal. To understand how a signal is produced, one must begin with an understanding of the beads. AlphaLISA® assays require two bead types: Donor beads and Acceptor beads. Each bead type contains a different proprietary mixture of chemicals. Donor beads contain a photosensitizer, phthalocyanine, which converts ambient oxygen to an excited and reactive form of O2, singlet oxygen, upon illumination at 680 nm. Note that singlet oxygen is not a radical; it is molecular oxygen with a single excited electron. Like other excited molecules, singlet oxygen has a limited lifetime prior to falling back to ground state. Within its 4 μsec half-life, singlet oxygen can diffuse approximately 200 nm in solution. If an Acceptor bead is within that proximity, energy is transferred from the singlet oxygen to thioxene derivatives within the Acceptor bead, subsequently culminating in light production at 520-620 nm. In the absence of an Acceptor bead, singlet oxygen falls to ground state and no signal is produced.

In one embodiment, the acceptor bead and the first antibody are linked by the interaction of streptavidin and biotin. In one embodiment, the acceptor bead is coated with streptavidin and the first antibody is linked to biotin. In one embodiment, the donor bead and the second antibody are linked by cyanoborohydride activation. In one embodiment, the first and the second antibody specifically bind to the detectable tag (T), which can be FLAG.

### Example 1

### Materials and Methods

cDNA Constructs and Cell Line Generation:

The assembly of this series of TAU expression constructs was based on Refseq NP_005901 (MAPT, “human 4R2N TAU”, SEQ ID NO:1) and truncated “K18” Tau (AA 244-372 of NP_005901, shown here as SEQ ID NO:7). The P2A amino acid sequence containing N-terminal GSG residues is as described in SEQ ID NO: 17. All additional amino acid substitutions and immuno-tags are as described. Corresponding nucleotide sequences were either native or codon optimized and assembled by gene synthesis (modified Gibson assembly) followed by subcloning into pcDNA3.1, pJTI-R4-DEST-CMV-pA or pJTI-R4-DEST-CMV_TO-pA (Life Technologies). Transient expression studies employed either pcDNA3.1 or pJTI-R4-DEST-CMV-pA vector backbones. Constitutive or tet-inducible stable cell lines were selected following co-transfection of full-length sequence verified TAU DNA+DNA encoding appropriate integrase enzyme into JumpIn_Hek293 or Trex_JumpIn_Hek293 parental cells from Invitrogen. All transfections utilized Fugene 6 transfection reagent (Promega). Selection media for the JumpIn_Hek293 stable lines included 10 μg/mL Blasticidin while 5 μg/mL Blasticidin+1000 μg/mL Geneticin was employed to select stable cells in the Trex_JumpIn_Hek293 background. Stable cell populations were subcloned by limiting dilution and clones were selected for tau expression in the induced state with minimal leakage in the un-induced state.

### Cell Induction and Lysis:

Stable Trex_JumpIn_HEK293 cell clones were isolated and grown under continuous antibiotic selection in DMEM containing 10% Dialyzed FBS. Tau constructs were expressed by induction with 1 ug/ml doxycycline for 3 to 5 days. Cells were harvested by lysis in PBS containing 1% triton X-100. Nuclei and debris were removed by centrifugation at 14,000 g and the supernatant was used for tau assays.

### AlphaLisa® Assays of Total Tau, Tau Phosphoepitopes and Tau Aggregates:

To overcome the limitations of Western Blot quantitation, the relative levels of tau phosphoepitopes were assessed using semi-quantitative AlphaLisa® immunoassays (Perkin Elmer). Briefly, HT7 (Thermo), DAKO anti-tau (DAKO corporation), or DA9 (courtesy of Peter Davies; see, d'Abramo et al., PLoS One. 2013 Apr. 29; 8(4):e62402) total tau antibodies were coupled to AlphaLisa beads by cyanoborohydride activation. Phosphoepitope-specific tau antibodies (from Thermo or from Dr. Davies) were biotinylated and bound to streptavidin-coated AlphaLisa donor beads. For each antibody pair utilized, sample lysates were individually titrated into the linear range of that assay. When samples from different cell lines were compared, total tau levels between the cells were normalized by titrating each of the respective lysates in a total tau assay consisting of HT7 acceptor beads, streptavidin donor beads and biotinylated BT2 (Thermo). Once normalized to total tau, the same ratio of lysates was maintained as those lysates were diluted pairwise into the linear range of each phospho-epitope immunoassay. The performance of the total tau assay was evaluated using a standard curve of bacterially-expressed human tau monomer. The sensitivity of the total tau assay enabled detection of tau standard between 10 pM and 3000 pM final tau concentration.

Detection of tau aggregates composed of tau+K18 (pseudo heterodimers) was performed using an AlphaLisa® assay consisting of monoclonal antibody HT7 on both sides of the immunoassay sandwich.

Detection of tau aggregates derived from FLAG-tagged tandem repeat tau (pseudo homodimers) used an AlphaLisa® assay consisting of FLAG antibody (Perkin Elmer and Cell Signaling) on both sides of the sandwich.

### Size Exclusion Chromatography:

Cells were grown on 150 mM plates and induced for 4 days by addition of 1 ug/ml of doxycycline. Culture media was removed and cells were quickly washed once with 20 ml of 10 mM Hepes pH 7.5. Lysates were prepared by extraction with 2 ml PhosphoSafe buffer and insoluble material was removed by centrifugation at 14,000 g for 10 minutes. The supernatant was concentrated 20-fold in a Amicon Ultra 15 concentrator (30 kDa cutoff) to achieve 10-15 mg/ml total protein. A 200 ul aliquot of concentrated cell lysate was applied to a Superose 6-10/300 FPLC column pre-equilibrated with 100 mM MES pH 6.9, 150 mM NaCl, and 1 mM DTT. Column fractions were assayed for total tau using 10 ul from a 1:100 dilution of the fractions. Tau oligomer immunoreactivity was assayed using 20 ul of undiluted fractions in the FLAG/FLAG assay format. Molecular size comparisons were made by running the same column with a series of protein standards ranging from 29,000 to 680,000 daltons. Protein elution was monitored by a UV absorbance detector at 280 and 254 nm.

### Detergent-Insoluble Tau:

Stable cells expressing tau monomer or tau tandem repeat were induced for 1 day or for 4 days and then extracted in PBS containing 1% Triton X-100 detergent. The extracts were cleared by an initial centrifugation at 14,000 g for 15 minutes. The pellet was discarded and then the cleared supernatants were centrifuged at 125,000 g for 40 minutes. High speed pellets were washed by resuspention in PBS containing 1% Sarkosyl and then centrifuged a second time at 125,000 g. The soluble supernatants from the low speed spin (S1), the first ultracentrifugation (S2) and the washed pellets (P3) were evaluated by Western blot using HT7 antibody.

### Preparation of Tau Oligomers In Vitro:

Recombinant His-tagged human 4R2N T40 was expressed by bacterial cultures. Cells were harvested following a 10 min centrifugation at 5,000×g. Cell paste was resuspended in 5× lysis buffer (50 mM Tris-HCl, 1 mM EGTA, 1 mM MgSO₄, 2 mM DTT, 750 mM NaCl, 20 mM NaF, 1 mM PMSF, 20 mM imidazole and proteinase inhibitor tablet), and lysed using a microfluidizer. The cell lysate was heat inactivated by immersion in boiling water (20 min at 95° C.), then crash cooled on wet ice. The inactivated lysate was then clarified by centrifugation at 40,000×g for 1 hour at 4° C. Tau protein purification was performed using a HisTrap FF column. The column was washed with buffer A (50 mM Tris-HCl, 1 mM EGTA, 1 mM MgSO₄, 2 mM DTT, 750 mM NaCl, 20 mM NaF, 1 mM PMSF, 20 mM imidazole) and eluted with buffer B (50 mM Tris-HCl, 1 mM EGTA, 1 mM MgSO₄, 2 mM DTT, 750 mM NaCl, 20 mM NaF, 1 mM PMSF, 500 mM imidazole). The resulting fractions with the highest tau content were pooled and formulated into 100 mM Na-acetate pH 7.0 using G25 desalting column. Aliquots of recombinant tau were snap-frozen and stored at −20° C. To prepare tau oligomers, 5 μM recombinant tau, dissolved in MES buffer (pH=6.5; 4-Morpholineethanesulfonic acid hydrate), was mixed with 10 μM DTT (BioShop) and incubated for 10 min at 55° C. Tau oligomer formation was induced via the addition of 5 μM heparin (Fisher, H19) and incubation with shaking (1000 rpm) for 4 h at 37° C. Tau fibrils were prepared through a similar protocol, but were allowed to aggregate with heparin at pH 7.0 for 14 days at 37° C. Tau monomers, used as a control in this study, were prepared through an identical protocol without the addition of heparin.

### MC1 Immunocytochemistry:

Cells were fixed in 4% paraformaldehyde for 15 min at room temperature (RT) followed by 3 washes with DPBS. Cells were then incubated in blocking and permeabilization buffer for 1 h shaking at room temperature [buffer: 0.2% Triton-X-100 (Sigma), 2% Goat serum (Sigma) and 0.1% BSA (Sigma) in DPBS (Sigma)]. After blocking, cells were incubated overnight at 4° C. with tau antibody MC-1 (kindly provided by Dr. Peter Davies) at 2 ug/ml in antibody solution (2% Goat serum, 0.1% BSA in DPBS). The day after, plates were washed 3 times (5 min each) with DPBS. Secondary antibodies diluted in antibody solution (1:600) were incubated for 1 hour at room temperature, and subsequently washed 3 times in DPBS. Hoechst 33342 solution was used to stain nuclei (Anaspec, 83218, 1:5000 in DPBS) (100 μl/well). High content imaging was performed using an Operetta system (PerkinElmer) with a 20× objective.

### Thioflavin-S Staining:

Cells were permeabilized with 0.1% TritonX-100 for 15 min in the presence of 4% paraformaldehyde and then were washed two times with PBS. Thioflavin-S 0.01% (ThS, Sigma) was added for 5 minutes at room temperature, followed by three 5-minute washes in 70% ethanol and final addition of PBS. High content imaging was performed using an Operetta system (PerkinElmer) with a 20× objective.

### Western Blot:

SDS-PAGE was performed on Novex 4-12% Bis-Tris gels in MES buffer and transferred to nitrocellulose by iBLOT (Invitrogen). Nitrocellulose membranes were blocked with Odyssey blocking buffer (Li-Cor) and incubated with tau primary antibodies. IR-Dye 800 secondary antibodies were used for detection on a LiCor Odyssey scanner.

### Example 2

### Assays for Detecting Tau Oligomers

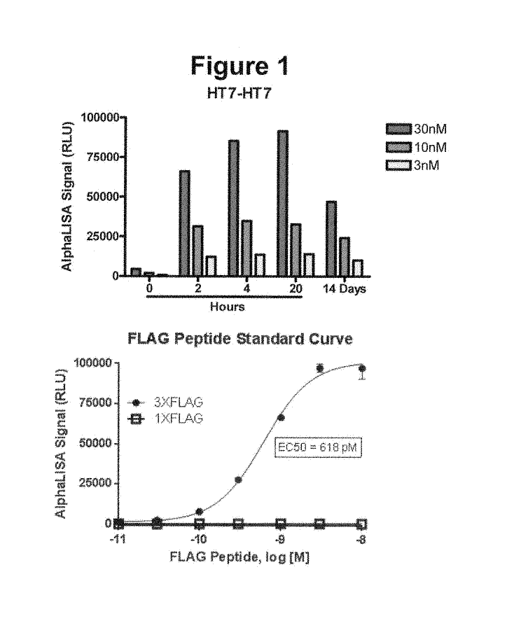

To enable rapid semi-quantitative detection of tau aggregates in cellular lysates we developed high throughput assays using bead-based AlphaLisa® immunoassays. AlphaLisa® technology involves the generation of singlet oxygen by light-induced excitation of an antibody-bound donor bead. When the donor bead is in close proximity to an antibody-bound acceptor bead the singlet oxygen triggers light release from the acceptor bead at a different wavelength. As binding of the antibody on one side of the sandwich obscures the recognition epitope, no assay signal can be generated unless two or more of the antibody recognition epitopes are closely associated. Two tau oligomer assays were initially validated against standards (FIG. 1). The first assay utilized antibody HT7, which recognizes an endogenous epitope in the human tau sequence between amino acids 159-163. An AlphaLisa assay was then developed to detect tau oligomers using HT7 on both donor and acceptor beads. The HT7/HT7 tau oligomer assay was initially tested against recombinant human tau monomers and tau oligomers formed in vitro. Assay signals were obtained within 2 hours of initiating aggregation with heparin and K18 repeat domain. The assay provided robust signals in the presence of tau aggregates equivalent to 300 pM to 3 nM of monomer (FIG. 1A). Only a minimal signal was elicited by 3 nM tau monomers that were not exposed to aggregating conditions. The assay also detected fibrillar tau formed by incubation for 14 days in vitro, but the signal was somewhat reduced by the formation of these larger aggregates compared to the smaller tau oligomers formed during shorter incubation times.

A second oligomer detection assay was subsequently developed using an antibody to the FLAG tag. The performance of the FLAG/FLAG oligomer assay was similarly tested using synthetic peptides comprised of 1×FLAG, representing a FLAG monomer sequence, or of 3× FLAG, to mimic multiple FLAG tags that would be present on recombinant tau oligomers containing a FLAG-tag (FIG. 1B). The use of 3×FLAG peptide resulted in a robust signal in the FLAG/FLAG oligomer assay in the range of 30 picomolar to 3000 picomolar of 3×FLAG peptide. No signal was obtained at any concentration of 1×FLAG peptide, supporting the specificity of the assay for detecting molecular species containing multiple copies of the FLAG epitope versus those containing only single copies of the tag.

### Example 3

### Size Exclusion of FPLC of Tau Oligomers

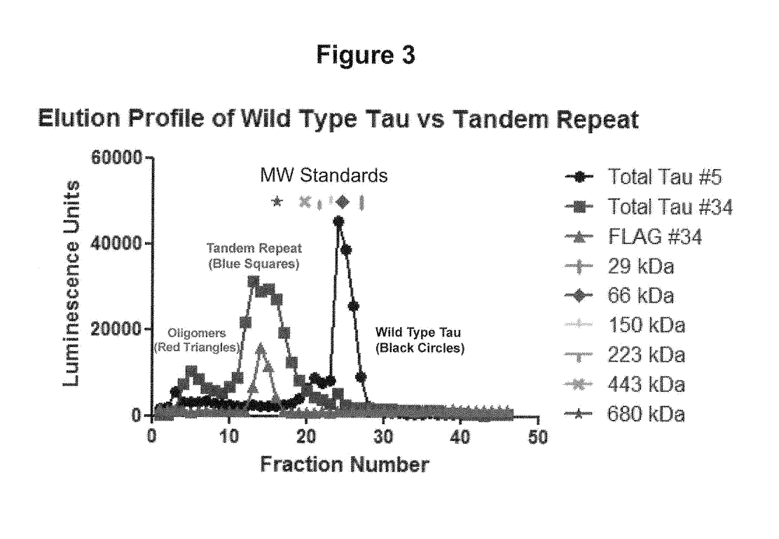

Confirmation that the tandem repeat tau construct was generating multimolecular complexes of tau was achieved by running cell lysates through Superose 6 size exclusion FPLC (10×300 mm column) and assaying fractions by AlphaLisa® immunoassays. The elution profile of full length tau monomer derived from a stable, inducible HEK293 cell line expressing wild type human tau monomer was compared to a series of protein standards, run separately (FIG. 3). Monomeric tau from HEK293 cells eluted in similar fractions as did the BSA standard (MW 66 kDa). At approximately twice the size, the tandem repeat tau protein would be expected to elute in the range between 90 to 130 kDa, which would be nearly indistinguishable from the elution profile of tau monomers in this column. However, total tau signal from tandem repeat tau-expressing cell lysates eluted much earlier than did tau monomer, and appeared to represent a broad range of molecular sizes between about 500 kDa to over 2000 kDa (overlayed onto FIG. 3). Analysis of these fractions in the FLAG/FLAG immunoassay confirmed that the high molecular weight tau complexes contained multiple copies of the FLAG tag.

### Example 3

### Time Course for Induction of Tau Expression, Hyperphosphorylation and Aggregation

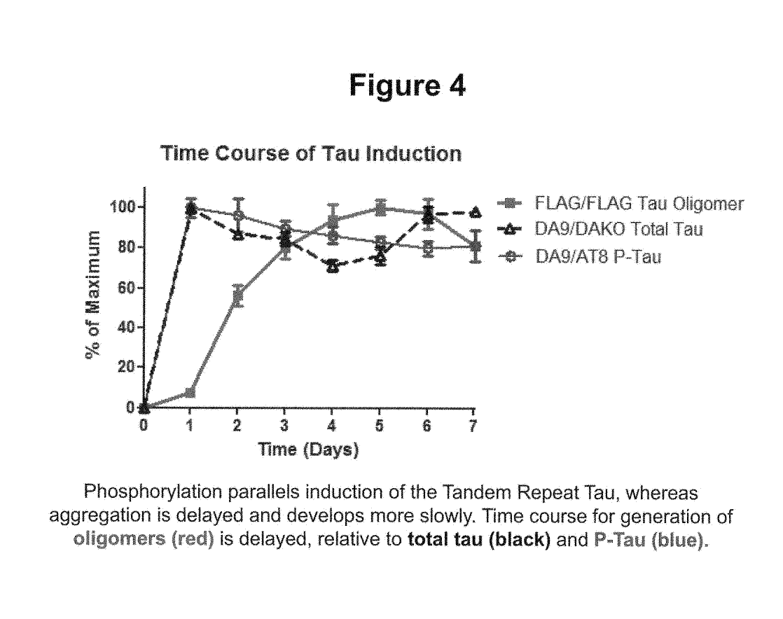

The time course of tau expression, phosphorylation, and aggregation was examined in a stable, inducible cell line expressing tandem repeat tau. Measurements of total tau, AT8 phospho-tau, and FLAG/FLAG oligomeric tau were performed by AlphaLisa® immunoassays of cell lysates at multiple time points after induction (FIG. 4). Total tau levels reached equilibrium by 24 hours after doxycycline induction and remained elevated for 7 days under continuous induction. Similarly, AT8 phosphotau levels also reached equilibrium by 24 hours and remained elevated. In contrast, the FLAG/FLAG immunoreactivity was barely detectable after 1 day, reached about 50% of maximum at 2 days, and continued to rise until the 5th day of continuous induction, suggesting that tandem repeat tau is initially expressed in a non-aggregated form and then progressively aggregates over time.

### Example 4

### Evaluation of Tau Phosphorylation at Multiple Epitopes

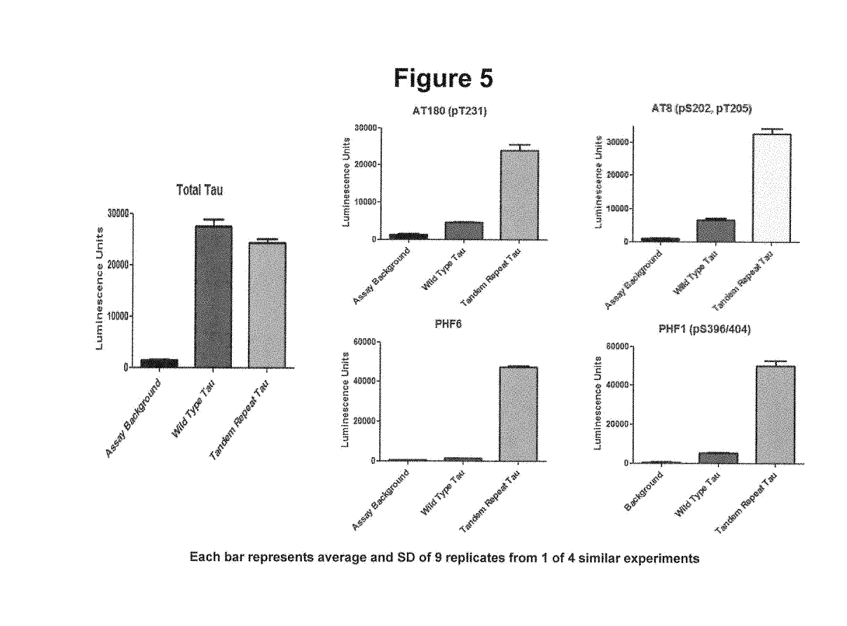

To compare the relative levels of tau phosphorylation between tau monomers and tandem repeat tau, lysates from stable inducible cell lines expressing monomer and tandem repeat constructs were normalized for total tau levels and evaluated in a series of phosphoepitope immunoassays. Because the tandem repeat construct contains twice as many antibody binding sites as tau monomer, it is not possible to directly compare molar amounts of this chimeric protein with a monomeric tau standard. Thus the lysate dilutions were adjusted to achieve equivalent AlphaLisa® signals in the total tau assay. This required a five-fold larger dilution factor for the tandem repeat tau cell lysates than for the tau monomer cell lysates. Subsequently, these lysates were diluted at this same ratio for AlphaLisa® measurements of phosphotau that were performed within the linear range of each phosphoepitope immunoassay. Comparisons of total tau immunoreactivities and those of 4 phosphoepitope antibodies are shown in FIG. 5. For each of these phosphoepitopes the immunoreactivity levels were 5 to 10 times higher in tandem repeat tau than in wild type tau monomer, despite the larger dilutions used for the tandem repeat tau cell lysates. Proteolytic digestion and Mass spectrometry of tandem repeat tau isolated from cell lysates confirmed that various individual tau peptides showed large increases in phosphorylation at multiple sites compared to the same peptides derived from tau monomer cell lysates (data not shown).

### Example 5

### Detection of Thioflavin-S and MC1 Positive Inclusions in HEK293 Cells Transiently Transfected with Tandem Repeat Tau

Transiently transfected HEK293 cells were examined for binding of Thioflavin-S and the conformation dependent MC1 tau antibody. HEK293 cells were transiently transfected with tau tandem repeat plasmid. Cells were fixed in 4% paraformaldehyde at 72 hours after transfection and incubated with the MC1 conformation-specific tau antibody and Thioflavin S. Image analysis showed co-localization of MC1-positive cells with Thioflavin-S binding (data not shown) in cells transfected with tandem repeat tau. Neither Thioflavin-S nor MC1 showed binding in cells transfected with wild type tau (not shown).

### Example 6

### Western Blots Showing Expression of Tandem Repeat Tau in the Total Lysate and in the Soluble and Detergent Insoluble Fractions

To determine whether expression of tandem repeat tau generates detergent-insoluble tau aggregates, triton extracts were subjected to differential ultracentrifugation and the soluble supernatant and insoluble pellet fractions were evaluated by Western blot using HT7 and FLAG antibodies. Tandem repeat tau was induced for one day or for four days prior to cell lysis in PBS with 1% triton X-100. Lysates were clarified by centrifugation at 14,000 g for 15 minutes. The 14,000 g supernatant 51 was then centrifuged at 125,000 g for 45 minutes and the supernatant S2 was sampled for analysis. The high speed pellet P2 was resuspended in triton lysis buffer and spun again at 125,000 g to generate pellet P3 for analysis. During the first day of induction a prominent 130 kDa band was present in the lysate (data not shown). Following ultracentrifugation, this band was found primarily in the high speed pellet. After 4 days induction, two additional prominent HT7-immunoreactive bands appeared in the total lysate, and precipitated in the high speed pellet. These bands migrated slightly above (60 kDa) and slightly below (55 kDa) that of tau monomer, indicating they resulted from a proteolytic cleavage event near the middle of the tandem repeat protein. The upper band from the two cleaved protein products in the high speed pellet, as well as the full-length uncleaved protein contained the FLAG tag, suggesting that proteolytic cleavage events may occur at both the N-terminal region and the C-terminal region of tau in the tandem repeat product.

### Example 7

### Detection of Tau Oligomers in HEK293 Cells Transiently Transfected with Various Tau Expression Plasmids

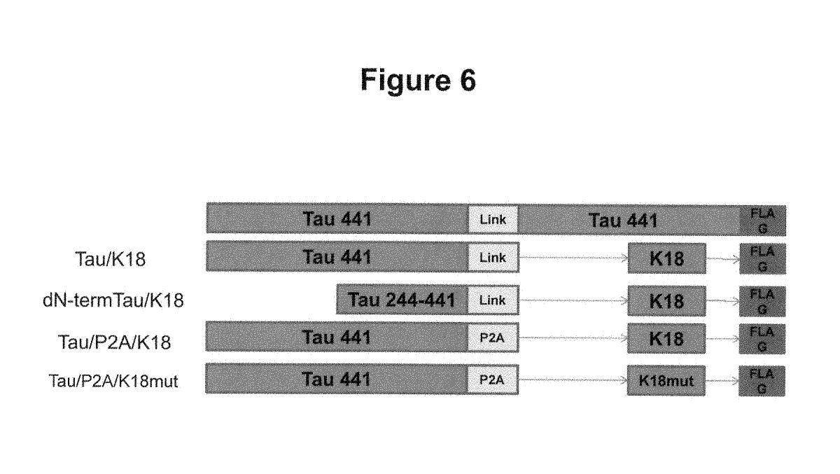

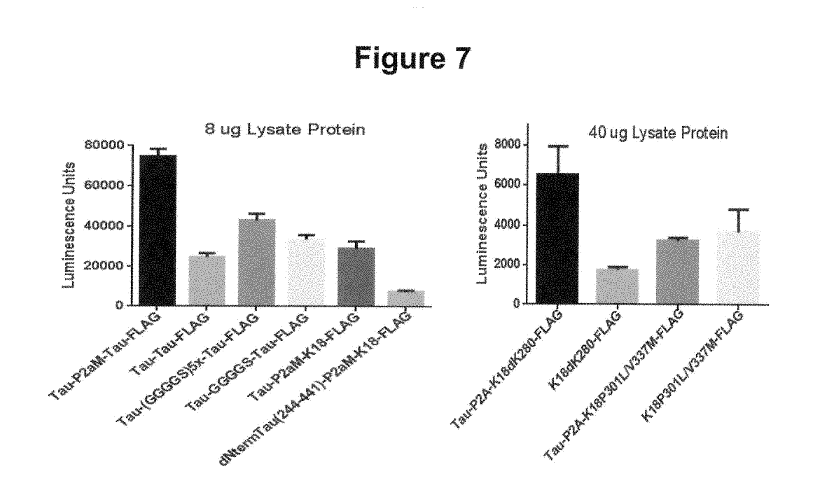

A series of FLAG tagged tau chimeric proteins were generated and transiently expressed in HEK293 cells to further delineate the molecular features required to promote cellular tau aggregation (FIG. 6). The role of the linker sequence between the two copies of tau was assessed by preparing constructs in which the 22 amino acid linker sequence (GSGATNFSLLKQAGDVEENAVP; SEQ ID NO:17) used to generate Tandem Repeat Tau was either removed entirely (Tau-Tau-FLAG; SEQ ID NO:12), was replaced by a five amino acid linker (Tau-QQQQS-Tau-FLAG; SEQ ID NO:13), or was replaced by a 25 amino acid linker (Tau-(QQQQS)5X-Tau-FLAG; SEQ ID NO:14). Evaluation of the protein products from these transfected sequences in the FLAG/FLAG aggregate assay revealed that the sequence or length of the linker region between the two copies of tau has only a small impact upon the protein aggregation. Both the shorter five amino acid QQQQS linker (SEQ ID NO:13) and the longer 25 amino acid 5X-QQQQS linker (SEQ ID NO:14) generated levels of aggregates only slightly less than the 22 amino acid linker. Aggregates were also generated following the complete removal of the linker region, albeit with somewhat reduced efficiency. These results suggesting that the specific linker sequence does not play a critical role in aggregate formation but a 22 amino acid linker may be marginally superior to shorter or longer linkers in supporting aggregation of tandem repeat tau constructs (FIG. 7). Evaluation of the pattern of protein cleavage products from the different constructs suggests that the cleavages are not occurring within the linker sequence.

Further analysis of the minimal molecular features of tau required for aggregate formation involved the use of truncated versions of the Tandem Repeat Tau. Constructs composed of one full length copy of tau coupled by the 22 amino acid linker region to the K18 fragment (Tau-P2aM-K18-FLAG; SEQ ID NO:15) were nearly as effective at generating tau aggregates as was Tandem Repeat Tau (FIG. 7). Despite the relatively robust aggregate formation by the Tau-P2aM-K18-FLAG construct, the N-terminal region of tau appears to play an important role in supporting aggregation, since N-terminal cleavage of the first 243 amino acids in the dNtermTau(244-441)-P2aM-K18-FLAG (SEQ ID NO:16) construct reduced aggregate formation considerably.

The covalent association of full length tau with the K18 fragment in these constructs appears to be critical to support rapid aggregation in this model system. The 22 amino acid linker sequence that we used to generate Tandem Repeat Tau or the Tau/Linker/K18 construct is based upon the P2AM sequence described by Osborn et al (39). Tau sequences containing the P2AM linker transcribe as a single mRNA and translate as a single parent protein. We also generated constructs in which two amino acids in the P2AM linker region were modified to generate a competent P2A picomavirus linker sequence. The competent P2A picomavirus sequence promotes translation of two separate proteins from a single mRNA strand through a process of ribosomal skipping (40, 41). Mutagenesis of the Tau/Linker/K18 construct to a Tau/P2A/K18 construct resulted in generation of separate tau and K18 proteins. Only a small fraction of the expressed protein from the P2A construct consisted of extended chimeric protein containing both tau and K18. The P2A-containing construct showed minimal aggregate formation, as assessed by either the FLAG/FLAG assay or by the HT7/HT7 assay. Similarly, two FLAG tagged K18 constructs containing Frontotemporal mutations were individually expressed and examined in the FLAG/FLAG aggregate assay. FLAG/FLAG immunoreactivity was observed for both of these constructs, but the levels of immununoreactivities were considerably reduced, compared to the Tau-Linker-K18-FLAG construct (compare FIG. 7).

### Example 8

### Incorporation of Wild Type Tau Monomers into Aggregates Formed by Tandem Repeat Tau

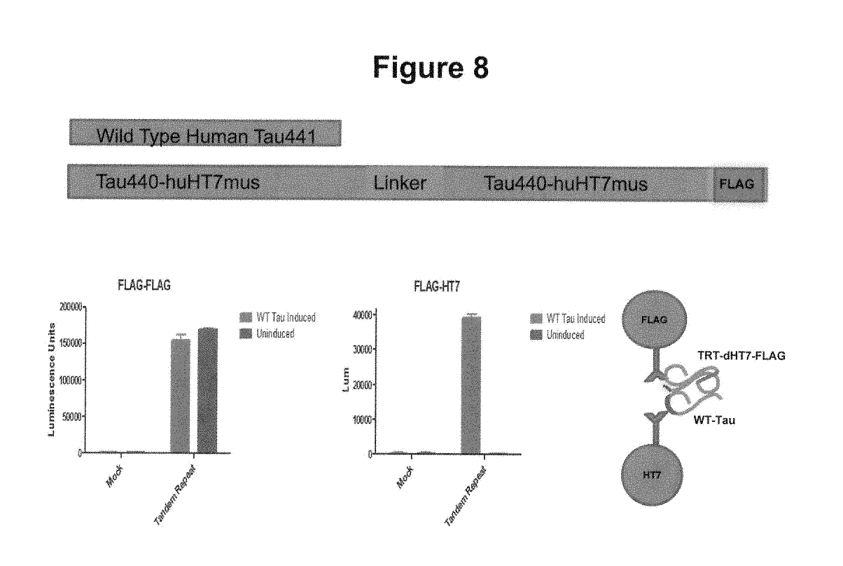

To determine whether tandem repeat tau can incorporate monomeric wild type human tau into aggregates, we performed co-expression studies and evaluated cell lysates by a modified protocol that was designed to detect mixed aggregates comprised of Tandem Repeat Tau and wild type tau monomer. We first generated a stable cell line that inducibly expresses wild type human tau with no FLAG tag on it. These wild type human tau cells were transiently transfected with a FLAG-tagged version of the Tandem Repeat Tau construct in which both of the HT7 recognition epitopes were mutated to the mouse sequence. The HT7 antibody did not recognize the protein product of this mutated tandem repeat construct as assessed by both AlphaLisa assay and Western blot (not shown). Similarly, the untagged monomeric tau did not bind the anti-FLAG antibody. Following transfection of tau monomer cells with the mutant tandem repeat tau construct, the cells were induced to express tau monomer and an AlphaLisa® assay was run using HT7 and FLAG antibodies. The detection of HT7/FLAG AlphaLisa® immunoreactivity in the transfected cells following monomer induction, but not in the absence of monomer induction (FIG. 8), supports that wild type tau monomer is able to be incorporated into the FLAG-tagged aggregates generated by tandem repeat tau.

### DISCUSSION

The cellular model of tau aggregation described here is distinct from previous models in that it does not require the use of Frontotemporal dementia mutations, it does not require seeding, and it utilizes the entire coding sequence of the longest isoform of human tau, expressed as two covalently coupled copies of wild type tau. This model spontaneously forms aggregates containing monomeric tau in a time-dependent manner. These tau aggregates share several characteristics with those formed in the brains of Alzheimer's patients, including hyperphosphorylation at several AD-relevant epitopes, binding to the beta sheet-binding agent Thioflavin-S, generation of multiple truncation products, and formation of detergent-insoluble aggregates. The spontaneous manner in which these post-translational events follow from the expression of a dimer-like form of tau suggests that the initial association event, whereby tau monomers create tau dimers, may be sufficient to provoke both the nucleation of tau aggregation into larger structures and the series of endogenous post-translational modifications that are associated with tau pathologies.

Tau C-terminal truncation products are highly prone to aggregation in vitro (57) and in neuronal expression models (58). Proteolytic generation of tau cleavage products has been proposed as an initiating factor for tau pathology in Alzheimer's disease (59-62). In the current cellular model, the tandem repeat tau construct is initially expressed as a full length protein, but numerous proteolytic cleavages occur rapidly and some of these cleavages give rise to two main products that are slightly larger and slightly smaller than bona fide tau monomer. These proteolytic products appear to concentrate in the detergent-insoluble fraction and their appearance coincides with the formation of aggregates. Here again, the generation of the dimer-like protein is the stimulus for the proteolytic events, since expression of tau monomer does not generate the same cleavage pattern. It is unlikely that cleavage within the linker region accounts for these additional protein products, since proteolysis within the internal linker should generate two protein bands that are each as large, or larger, than full length tau. The pattern of proteolytic products was similar in constructs containing linker regions with different lengths and different sequences, or in a construct containing no linker region

In an effort to define the molecular features required for aggregate formation, we generated a series of modified constructs and compared their abilities to aggregate. When one copy of full length tau was coupled to one copy of K18, the protein product aggregated to a similar extent as did the full length tandem repeat tau product. However, mutation of the linker region to the competent picornavirus P2A sequence led to translation of separate tau and K18 monomers in an equi-molar ratio. Cellular expression of these two non-linked proteins generated much less signal in our assay than did expression of the covalently linked hybrid protein. Thus in this model system the mere co-expression of tau with K18 repeat domains containing Frontotemporal dementia mutations did not induce comparable aggregate formation to that from the covalently coupled tau/K18 construct. We also examined aggregation of C-terminally FLAG-tagged K18(dK280) or K18(P301LN337M) using the FLAG/FLAG AlphaLisa® assay. Similarly to the Tau-P2A-K18-FLAG constructs, these K18 mutant constructs also gave low signals in the FLAG/FLAG assay. These results were unexpected and might appear to contrast with the mutant K18 cellular expression studies of Khlistunova et al (31) and of Kfoury et al (32) and also to contrast with the recent report of Harrington et al (63) who co-expressed full length tau with tau repeat domain regions. Some of the apparent differences in our findings versus these other reports likely relate to the different methods used for detection of tau aggregates. Kfoury (32) used HEK293 cells to express mutant tau repeat domain (RD) coupled to fluorescent proteins (FP) and then monitored RD-FP aggregation with FRET. Khlistunova (31) expressed mutant K18 in N2a cells and then detected aggregates using thioflavin-S fluorescence. Similarly, Harrington (63) used primulin fluorescence to detect tau aggregates in 3T6H fibroblasts co-expressing tau and repeat domain. The fluorescent detection methods used in these intact cellular model systems are likely more sensitive in the detection of low levels of oligomers than the AlphaLISA assay we employed here for detection of tau aggregates in detergent lysates. Shammas et al (64) have recently reported an extensive kinetic analysis of K18 oligomer formation in vitro using FRET analysis of fluorescently-tagged K18. They note that under conditions where oligomer formation is monitored prior to fibril formation, Thioflavin T fluorescence reaches equilibrium within 3 hours of initiating oligomer formation, but oligomers represent only 0.1% of the total amount of tau present and are comprised of low copy number assemblies. Following dilution in aqueous buffer, these K18 oligomers rapidly dissociate. Thus, detection of cellular K18 oligomers by Thioflavin S fluorescence must be performed in fixed cells and is not readily detected in cellular lysates. Our data suggest that tandem repeat tau and covalently coupled tau-K18 are considerably more efficient than either K18 alone or K18 co-expressed with full length tau in the generation of stable tau aggregates that survive cellular lysis. These tandem repeat tau aggregates may either be present in larger number or they may be larger in size than aggregates formed from mutant K18 repeat domains, thus allowing more robust detection by the AlphaLisa assays used here.

As part of our characterization of structural requirements necessary for tau aggregation, we have confirmed a previously recognized critical role of the N-terminus of tau in tau aggregate formation (65). Deletion of the N-terminal 243 amino acids from a tau/K18 construct greatly reduced aggregate formation in this cellular model. The mere presence of two coupled C-terminal repeat domains in the dNtermTau-Linker-K18 molecule does not match the aggregate-promoting properties of Tau-Linker-K18.

While it is not obvious why expression of a covalently linked Tau-Linker-K18 construct or of a full length tandem repeat tau construct should induce robust tau aggregation when equi-molar expression of each of the individual components does not induce appreciable aggregation, two factors may contribute to this outcome. The first factor is the impact of a covalent linkage on the relative tau concentration within the microenvironment. In vitro studies of tau aggregation have demonstrated that the rate of aggregation is strongly impacted by the tau concentration in solution. Covalently linking two copies of tau effectively increases the local concentration of tau molecules, relative to non-coupled copies of the protein. This relative concentration effect may force the tandem repeat protein to adopt a dimer-like conformation that nucleates the aggregation progression. The second factor is that of charge interactions. Tau is a highly charged and natively unfolded protein containing a basic, positively charged core, sandwiched between acidic, negatively charged N- and C-terminal regions (66, 67). Models of tau conformation have described the secondary structure as either that like a paperclip, where the acidic C-terminal end folds over the basic core and comes into close proximity of the acidic N-terminal region (68), or alternatively as that of a naturally-occurring dimer with antiparallel stacking in a manner that aligns the positively charged core of each molecule with the negatively charged N-terminus of the complementary molecule (69). If ionic bonding is involved in stabilizing the dimer-like conformation of tandem repeat tau to promote aggregation, then modulation of the length or sequence of the intervening linker region may have minimal impact on the ability of the tandem repeat arrangement to achieve the nucleating conformation. We have examined the impact of several linkers on the ability of the tandem repeat tau construct to form tau aggregates. Tau aggregates were generated to a similar extent using four different linkers ranging in size between 0 to 25 amino acids in length. These gene products also underwent similar post-translational processing, indicating that the critical conformation leading to aggregation is not dependent upon properties introduced by the linker.

These initial studies of the tandem repeat tau construct demonstrate that a progressive cascade of events, which includes tau hyperphosphorylation, proteolytic cleavage, and aggregation, spontaneously follows the expression of this dimer-like tau construct. These results suggest that the initial association of tau monomers into tau dimers may be sufficient to initiate the nucleation of tau aggregates and to provoke a series of endogenous cellular processes associated with the development of tau pathologies. While we have also been able to demonstrate that these same events occur when this construct is expressed in neurons (to be reported elsewhere), the basic cellular processes that result in these disease-relevant post-translational processing events are not specific to post-mitotic neurons, as has been shown here with proliferating HEK293 cells.

SEQ ID

| NO: | Description | Sequences |
|---|---|---|
| SEQ ID | Human Tau | MAEPRQEFEV MEDHAGTYGL GDRKDQGGYT |
| NO: 1 | 4R2N | MHQDQEGDTD AGLKESPLQT PTEDGSEEPG SETSDAKSTP |
| | | TAEDVTAPLV DEGAPGKQAA AQPHTEIPEG TTAEEAGIGD |
| | | TPSLEDEAAG HVTQARMVSK SKDGTGSDDK |
| | | KAKGADGKTK IATPRGAAPP GQKGQANATR |
| | | IPAKTPPAPK TPPSSGEPPK SGDRSGYSSP GSPGTPGSRS |
| | | RTPSLPTPPT REPKKVAVVR TPPKSPSSAK SRLQTAPVPM |
| | | PDLKNVKSKI GSTENLKHQP GGGKVQIINK KLDLSNVQSK |
| | | CGSKDNIKHV PGGGSVQIVY KPVDLSKVTS KCGSLGNIHH |
| | | KPGGGQVEVK SEKLDFKDRV QSKIGSLDNI |
| | | THVPGGGNKK IETHKLTFRE NAKAKTDHGA |
| | | EIVYKSPVVS GDTSPRHLSN VSSTGSIDMV DSPQLATLAD |
| | | EVSASLAKQG L |
| 2 | Human Tau | MAEPRQEFEV MEDHAGTYGL GDRKDQGGYT |
| | 4R/1N | MHQDQEGDTD AGLKESPLQT PTEDGSEEPG SETSDAKSTP |
| | | TAEAEEAGIG DTPSLEDEAA GHVTQARMVS |
| | | KSKDGTGSDD KKAKGADGKT KIATPRGAAP |
| | | PGQKGQANAT RIPAKTPPAP KTPPSSGEPP KSGDRSGYSS |
| | | PGSPGTPGSR SRTPSLPTPP TREPKKVAVV RTPPKSPSSA |
| | | KSRLQTAPVP MPDLKNVKSK IGSTENLKHQ PGGGKVQIIN |
| | | KKLDLSNVQS KCGSKDNIKH VPGGGSVQIV |
| | | YKPVDLSKVT SKCGSLGNIH HKPGGGQVEV |
| | | KSEKLDFKDR VQSKIGSLDN ITHVPGGGNK KIETHKLTFR |
| | | ENAKAKTDHG AEIVYKSPVV SGDTSPRHLS NVSSTGSIDM |
| | | VDSPQLATLA DEVSASLAKQ GL |
| 3 | Human Tau | MAEPRQEFEV MEDHAGTYGL GDRKDQGGYT |
| | 4R/0N | MHQDQEGDTD AGLKAEEAGI GDTPSLEDEA |
| | | AGHVTQARMV SKSKDGTGSD DKKAKGADGK |
| | | TKIATPRGAA PPGQKGQANA TRIPAKTPPA PKTPPSSGEP |
| | | PKSGDRSGYS SPGSPGTPGS RSRTPSLPTP PTREPKKVAV |
| | | VRTPPKSPSS AKSRLQTAPV PMPDLKNVKS KIGSTENLKH |
| | | QPGGGKVQII NKKLDLSNVQ SKCGSKDNIK HVPGGGSVQI |
| | | VYKPVDLSKV TSKCGSLGNI HHKPGGGQVE |
| | | VKSEKLDFKD RVQSKIGSLD NITHVPGGGN KKIETHKLTF |
| | | RENAKAKTDH GAEIVYKSPV VSGDTSPRHL SNVSSTGSID |
| | | MVDSPQLATL ADEVSASLAK QGL |
| 4 | Human Tau | MAEPRQEFEV MEDHAGTYGL GDRKDQGGYT |
| | 3R/2N | MHQDQEGDTD AGLKESPLQT PTEDGSEEPG SETSDAKSTP |
| | | TAEDVTAPLV DEGAPGKQAA AQPHTEIPEG TTAEEAGIGD |
| | | TPSLEDEAAG HVTQARMVSK SKDGTGSDDK |
| | | KAKGADGKTK IATPRGAAPP GQKGQANATR |
| | | IPAKTPPAPK TPPSSGEPPK SGDRSGYSSP GSPGTPGSRS |
| | | RTPSLPTPPT REPKKVAVVR TPPKSPSSAK SRLQTAPVPM |
| | | PDLKNVKSKI GSTENLKHQP GGGKVQIVYK |
| | | PVDLSKVTSK CGSLGNIHHK PGGGQVEVKS |
| | | EKLDFKDRVQ SKIGSLDNIT HVPGGGNKKI ETHKLTFREN |
| | | AKAKTDHGAE IVYKSPVVSG DTSPRHLSNV |
| | | SSTGSIDMVD SPQLATLADE VSASLAKQGL |
| 5 | Human Tau | MAEPRQEFEV MEDHAGTYGL GDRKDQGGYT |
| | 3R/1N | MHQDQEGDTD AGLKESPLQT PTEDGSEEPG SETSDAKSTP |
| | | TAEAEEAGIG DTPSLEDEAA GHVTQARMVS |
| | | KSKDGTGSDD KKAKGADGKT KIATPRGAAP |
| | | PGQKGQANAT RIPAKTPPAP KTPPSSGEPP KSGDRSGYSS |
| | | PGSPGTPGSR SRTPSLPTPP TREPKKVAVV RTPPKSPSSA |
| | | KSRLQTAPVP MPDLKNVKSK IGSTENLKHQ |
| | | PGGGKVQIVY KPVDLSKVTS KCGSLGNIFIH |
| | | KPGGGQVEVK SEKLDFKDRV QSKIGSLDNI |
| | | THVPGGGNKK IETHKLTFRE NAKAKTDHGA |
| | | EIVYKSPVVS GDTSPRHLSN VSSTGSIDMV DSPQLATLAD |
| | | EVSASLAKQG L |
| 6 | Human Tau | MAEPRQEFEV MEDHAGTYGL GDRKDQGGYT |
| | 3R/0N | MHQDQEGDTD AGLKAEEAGI GDTPSLEDEA |
| | | AGHVTQARMV SKSKDGTGSD DKKAKGADGK |
| | | TKIATPRGAA PPGQKGQANA TRIPAKTPPA PKTPPSSGEP |
| | | PKSGDRSGYS SPGSPGTPGS RSRTPSLPTP PTREPKKVAV |
| | | VRTPPKSPSS AKSRLQTAPV PMPDLKNVKS KIGSTENLKH |
| | | QPGGGKVQIV YKPVDLSKVT SKCGSLGNIH |
| | | HKPGGGQVEV KSEKLDFKDR VQSKIGSLDN |
| | | ITHVPGGGNK KIETHKLTFR ENAKAKTDHG AEIVYKSPVV |
| | | SGDTSPRHLS NVSSTGSIDM VDSPQLATLA DEVSASLAKQ |
| | | GL |
| 7 | K18 repeat | QTAPVPM PDLKNVKSKI GSTENLKHQP GGGKVQIINK |
| | domain of | KLDLSNVQSK CGSKDNIKHV PGGGSVQIVY |
| | human Tau (AA | KPVDLSKVTS KCGSLGNIHH KPGGGQVEVK |
| | 244-372 of SEQ | SEKLDFKDRV QSKIGSLDNI THVPGGGNKK IE |
| | ID NO: 1) | |
| 8 | Human Tau | MAEPRQEFEV MEDHAGTYGL GDRKDQGGYT |
| | P301L mutant | MHQDQEGDTD AGLKESPLQT PTEDGSEEPG SETSDAKSTP |
| | | TAEDVTAPLV DEGAPGKQAA AQPHTEIPEG TTAEEAGIGD |
| | | TPSLEDEAAG HVTQARMVSK SKDGTGSDDK |
| | | KAKGADGKTK IATPRGAAPP GQKGQANATR |
| | | IPAKTPPAPK TPPSSGEPPK SGDRSGYSSP GSPGTPGSRS |
| | | RTPSLPTPPT REPKKVAVVR TPPKSPSSAK SRLQTAPVPM |
| | | PDLKNVKSKI GSTENLKHQP GGGKVQIINK KLDLSNVQSK |
| | | CGSKDNIKHV LGGGSVQIVY KPVDLSKVTS |
| | | KCGSLGNIHH KPGGGQVEVK SEKLDFKDRV QSKIGSLDNI |
| | | THVPGGGNKK IETHKLTFRE NAKAKTDHGA |
| | | EIVYKSPVVS GDTSPRHLSN VSSTGSIDMV DSPQLATLAD |
| | | EVSASLAKQG L |
| 9 | Human wild | MAEPRQEFEV MEDHAGTYGL GDRKDQGGYT |
| | type tau-FLAG | MHQDQEGDTD AGLKESPLQT PTEDGSEEPG SETSDAKSTP |
| | | TAEDVTAPLV DEGAPGKQAA AQPHTEIPEG TTAEEAGIGD |
| | | TPSLEDEAAG HVTQARMVSK SKDGTGSDDK |
| | | KAKGADGKTK IATPRGAAPP GQKGQANATR |
| | | IPAKTPPAPK TPPSSGEPPK SGDRSGYSSP GSPGTPGSRS |
| | | RTPSLPTPPT REPKKVAVVR TPPKSPSSAK SRLQTAPVPM |
| | | PDLKNVKSKI GSTENLKHQP GGGKVQIINK KLDLSNVQSK |
| | | CGSKDNIKHV PGGGSVQIVY KPVDLSKVTS KCGSLGNIHH |
| | | KPGGGQVEVK SEKLDFKDRV QSKIGSLDNI |
| | | THVPGGGNKK IETHKLTFRE NAKAKTDHGA |
| | | EIVYKSPVVS GDTSPRHLSN VSSTGSIDMV DSPQLATLAD |
| | | EVSASLAKQG L DYKDDDDK |
| 10 | Human P301L | MAEPRQEFEV MEDHAGTYGL GDRKDQGGYT |
| | mutant tau- | MHQDQEGDTD AGLKESPLQT PTEDGSEEPG SETSDAKSTP |
| | FLAG | TAEDVTAPLV DEGAPGKQAA AQPHTEIPEG TTAEEAGIGD |
| | | TPSLEDEAAG HVTQARMVSK SKDGTGSDDK |
| | | KAKGADGKTK IATPRGAAPP GQKGQANATR |
| | | IPAKTPPAPK TPPSSGEPPK SGDRSGYSSP GSPGTPGSRS |
| | | RTPSLPTPPT REPKKVAVVR TPPKSPSSAK SRLQTAPVPM |
| | | PDLKNVKSKI GSTENLKHQP GGGKVQIINK KLDLSNVQSK |
| | | CGSKDNIKHV LGGGSVQIVY KPVDLSKVTS |
| | | KCGSLGNIHH KPGGGQVEVK SEKLDFKDRV QSKIGSLDNI |
| | | THVPGGGNKK IETHKLTFRE NAKAKTDHGA |
| | | EIVYKSPVVS GDTSPRHLSN VSSTGSIDMV DSPQLATLAD |
| | | EVSASLAKQG L DYKDDDDK |
| 11 | Tau tandem | MAEPRQEFEV MEDHAGTYGL GDRKDQGGYT |
| | repeat | MHQDQEGDTD AGLKESPLQT PTEDGSEEPG SETSDAKSTP |
| | polypeptide- | TAEDVTAPLV DEGAPGKQAA AQPHTEIPEG TTAEEAGIGD |
| | TAU-LINKER- | TPSLEDEAAG HVTQARMVSK SKDGTGSDDK |
| | TAU-FLAG | KAKGADGKTK IATPRGAAPP GQKGQANATR |
| | | IPAKTPPAPK TPPSSGEPPK SGDRSGYSSP GSPGTPGSRS |
| | | RTPSLPTPPT REPKKVAVVR TPPKSPSSAK SRLQTAPVPM |
| | | PDLKNVKSKI GSTENLKHQP GGGKVQIINK KLDLSNVQSK |
| | | CGSKDNIKHV PGGGSVQIVY KPVDLSKVTS KCGSLGNIHH |
| | | KPGGGQVEVK SEKLDFKDRV QSKIGSLDNI |
| | | THVPGGGNKK IETHKLTFRE NAKAKTDHGA |
| | | EIVYKSPVVS GDTSPRHLSN VSSTGSIDMV DSPQLATLAD |
| | | EVSASLAKQG L GSGATNFSLLKQAGDVEENAVP |
| | | MAEPRQEFEV MEDHAGTYGL GDRKDQGGYT |
| | | MHQDQEGDTD AGLKESPLQT PTEDGSEEPG SETSDAKSTP |
| | | TAEDVTAPLV DEGAPGKQAA AQPHTEIPEG TTAEEAGIGD |
| | | TPSLEDEAAG HVTQARMVSK SKDGTGSDDK |
| | | KAKGADGKTK IATPRGAAPP GQKGQANATR |
| | | IPAKTPPAPK TPPSSGEPPK SGDRSGYSSP GSPGTPGSRS |
| | | RTPSLPTPPT REPKKVAVVR TPPKSPSSAK SRLQTAPVPM |
| | | PDLKNVKSKI GSTENLKHQP GGGKVQIINK KLDLSNVQSK |
| | | CGSKDNIKHV PGGGSVQIVY KPVDLSKVTS KCGSLGNIHH |
| | | KPGGGQVEVK SEKLDFKDRV QSKIGSLDNI |
| | | THVPGGGNKK IETHKLTFRE NAKAKTDHGA |
| | | EIVYKSPVVS GDTSPRHLSN VSSTGSIDMV DSPQLATLAD |
| | | EVSASLAKQG L DYKDDDDK |
| 12 | Tau tandem | MAEPRQEFEV MEDHAGTYGL GDRKDQGGYT |
| | repeat | MHQDQEGDTD AGLKESPLQT PTEDGSEEPG SETSDAKSTP |
| | polypeptide- | TAEDVTAPLV DEGAPGKQAA AQPHTEIPEG TTAEEAGIGD |
| | TAU-TAU- | TPSLEDEAAG HVTQARMVSK SKDGTGSDDK |
| | FLAG | KAKGADGKTK IATPRGAAPP GQKGQANATR |
| | | IPAKTPPAPK TPPSSGEPPK SGDRSGYSSP GSPGTPGSRS |
| | | RTPSLPTPPT REPKKVAVVR TPPKSPSSAK SRLQTAPVPM |
| | | PDLKNVKSKI GSTENLKHQP GGGKVQIINK KLDLSNVQSK |
| | | CGSKDNIKHV PGGGSVQIVY KPVDLSKVTS KCGSLGNIHH |
| | | KPGGGQVEVK SEKLDFKDRV QSKIGSLDNI |
| | | THVPGGGNKK IETHKLTFRE NAKAKTDHGA |
| | | EIVYKSPVVS GDTSPRHLSN VSSTGSIDMV DSPQLATLAD |
| | | EVSASLAKQG LMAEPRQEFEV MEDHAGTYGL |
| | | GDRKDQGGYT MHQDQEGDTD AGLKESPLQT |
| | | PTEDGSEEPG SETSDAKSTP TAEDVTAPLV DEGAPGKQAA |
| | | AQPHTEIPEG TTAEEAGIGD TPSLEDEAAG HVTQARMVSK |
| | | SKDGTGSDDK KAKGADGKTK IATPRGAAPP |
| | | GQKGQANATR IPAKTPPAPK TPPSSGEPPK SGDRSGYSSP |
| | | GSPGTPGSRS RTPSLPTPPT REPKKVAVVR TPPKSPSSAK |
| | | SRLQTAPVPM PDLKNVKSKI GSTENLKHQP GGGKVQIINK |
| | | KLDLSNVQSK CGSKDNIKHV PGGGSVQIVY |
| | | KPVDLSKVTS KCGSLGNIHH KPGGGQVEVK |
| | | SEKLDFKDRV QSKIGSLDNI THVPGGGNKK IETHKLTFRE |
| | | NAKAKTDHGA EIVYKSPVVS GDTSPRHLSN |
| | | VSSTGSIDMV DSPQLATLAD EVSASLAKQG L |
| | | DYKDDDDK |
| 13 | Tau tandem | MAEPRQEFEV MEDHAGTYGL GDRKDQGGYT |
| | repeat | MHQDQEGDTD AGLKESPLQT PTEDGSEEPG SETSDAKSTP |
| | polypeptide- | TAEDVTAPLV DEGAPGKQAA AQPHTEIPEG TTAEEAGIGD |
| | Tau-QQQQS- | TPSLEDEAAG HVTQARMVSK SKDGTGSDDK |
| | Tau-FLAG | KAKGADGKTK IATPRGAAPP GQKGQANATR |
| | | IPAKTPPAPK TPPSSGEPPK SGDRSGYSSP GSPGTPGSRS |
| | | RTPSLPTPPT REPKKVAVVR TPPKSPSSAK SRLQTAPVPM |
| | | PDLKNVKSKI GSTENLKHQP GGGKVQIINK KLDLSNVQSK |
| | | CGSKDNIKHV PGGGSVQIVY KPVDLSKVTS KCGSLGNIHH |
| | | KPGGGQVEVK SEKLDFKDRV QSKIGSLDNI |
| | | THVPGGGNKK IETHKLTFRE NAKAKTDHGA |
| | | EIVYKSPVVS GDTSPRHLSN VSSTGSIDMV DSPQLATLAD |
| | | EVSASLAKQG LQQQQS MAEPRQEFEV MEDHAGTYGL |
| | | GDRKDQGGYT MHQDQEGDTD AGLKESPLQT |
| | | PTEDGSEEPG SETSDAKSTP TAEDVTAPLV DEGAPGKQAA |
| | | AQPHTEIPEG TTAEEAGIGD TPSLEDEAAG HVTQARMVSK |
| | | SKDGTGSDDK KAKGADGKTK IATPRGAAPP |
| | | GQKGQANATR IPAKTPPAPK TPPSSGEPPK SGDRSGYSSP |
| | | GSPGTPGSRS RTPSLPTPPT REPKKVAVVR TPPKSPSSAK |
| | | SRLQTAPVPM PDLKNVKSKI GSTENLKHQP GGGKVQIINK |
| | | KLDLSNVQSK CGSKDNIKHV PGGGSVQIVY |
| | | KPVDLSKVTS KCGSLGNIHH KPGGGQVEVK |
| | | SEKLDFKDRV QSKIGSLDNI THVPGGGNKK IETHKLTFRE |
| | | NAKAKTDHGA EIVYKSPVVS GDTSPRHLSN |
| | | VSSTGSIDMV DSPQLATLAD EVSASLAKQG L |
| | | DYKDDDDK |
| 14 | Tau tandem | MAEPRQEFEV MEDHAGTYGL GDRKDQGGYT |
| | repeat | MHQDQEGDTD AGLKESPLQT PTEDGSEEPG SETSDAKSTP |
| | polypeptide- | TAEDVTAPLV DEGAPGKQAA AQPHTEIPEG TTAEEAGIGD |
| | Tau- | TPSLEDEAAG HVTQARMVSK SKDGTGSDDK |
| | (QQQQS)5X- | KAKGADGKTK IATPRGAAPP GQKGQANATR |
| | Tau-FLAG | IPAKTPPAPK TPPSSGEPPK SGDRSGYSSP GSPGTPGSRS |
| | | RTPSLPTPPT REPKKVAVVR TPPKSPSSAK SRLQTAPVPM |
| | | PDLKNVKSKI GSTENLKHQP GGGKVQIINK KLDLSNVQSK |
| | | CGSKDNIKHV PGGGSVQIVY KPVDLSKVTS KCGSLGNIHH |
| | | KPGGGQVEVK SEKLDFKDRV QSKIGSLDNI |
| | | THVPGGGNKK IETHKLTFRE NAKAKTDHGA |
| | | EIVYKSPVVS GDTSPRHLSN VSSTGSIDMV DSPQLATLAD |
| | | EVSASLAKQGL QQQQSQQQQSQQQQSQQQQSQQQQS |
| | | MAEPRQEFEV MEDHAGTYGL GDRKDQGGYT |
| | | MHQDQEGDTD AGLKESPLQT PTEDGSEEPG SETSDAKSTP |
| | | TAEDVTAPLV DEGAPGKQAA AQPHTEIPEG TTAEEAGIGD |
| | | TPSLEDEAAG HVTQARMVSK SKDGTGSDDK |
| | | KAKGADGKTK IATPRGAAPP GQKGQANATR |
| | | IPAKTPPAPK TPPSSGEPPK SGDRSGYSSP GSPGTPGSRS |
| | | RTPSLPTPPT REPKKVAVVR TPPKSPSSAK SRLQTAPVPM |
| | | PDLKNVKSKI GSTENLKHQP GGGKVQIINK KLDLSNVQSK |
| | | CGSKDNIKHV PGGGSVQIVY KPVDLSKVTS KCGSLGNIHH |
| | | KPGGGQVEVK SEKLDFKDRV QSKIGSLDNI |
| | | THVPGGGNKK IETHKLTFRE NAKAKTDHGA |
| | | EIVYKSPVVS GDTSPRHLSN VSSTGSIDMV DSPQLATLAD |
| | | EVSASLAKQG L DYKDDDDK |
| 15 | Tau tandem | MAEPRQEFEV MEDHAGTYGL GDRKDQGGYT |
| | repeat | MHQDQEGDTD AGLKESPLQT PTEDGSEEPG SETSDAKSTP |
| | polypeptide- | TAEDVTAPLV DEGAPGKQAA AQPHTEIPEG TTAEEAGIGD |
| | Tau-P2aM-K18- | TPSLEDEAAG HVTQARMVSK SKDGTGSDDK |
| | FLAG | KAKGADGKTK IATPRGAAPP GQKGQANATR |
| | | IPAKTPPAPK TPPSSGEPPK SGDRSGYSSP GSPGTPGSRS |
| | | RTPSLPTPPT REPKKVAVVR TPPKSPSSAK SRLQTAPVPM |
| | | PDLKNVKSKI GSTENLKHQP GGGKVQIINK KLDLSNVQSK |
| | | CGSKDNIKHV PGGGSVQIVY KPVDLSKVTS KCGSLGNIHH |
| | | KPGGGQVEVK SEKLDFKDRV QSKIGSLDNI |
| | | THVPGGGNKK IETHKLTFRE NAKAKTDHGA |
| | | EIVYKSPVVS GDTSPRHLSN VSSTGSIDMV DSPQLATLAD |
| | | EVSASLAKQG L GSGATNFSLLKQAGDVEENAVP |
| | | QTAPVPM PDLKNVKSKI GSTENLKHQP GGGKVQIINK |
| | | KLDLSNVQSK CGSKDNIKHV PGGGSVQIVY |
| | | KPVDLSKVTS KCGSLGNIHH KPGGGQVEVK |
| | | SEKLDFKDRV QSKIGSLDNI THVPGGGNKK IE |
| | | DYKDDDDK |
| 16 | dNtermTau (244- | QTAPVPM PDLKNVKSKI GSTENLKHQP GGGKVQIINK |
| | 441)-P2aM- | KLDLSNVQSK CGSKDNIKHV PGGGSVQIVY |
| | K18-FLAG | KPVDLSKVTS KCGSLGNIHH KPGGGQVEVK |
| | | SEKLDFKDRV QSKIGSLDNI THVPGGGNKK IETHKLTFRE |
| | | NAKAKTDHGA EIVYKSPVVS GDTSPRHLSN |
| | | VSSTGSIDMV DSPQLATLAD EVSASLAKQG L |
| | | GSGATNFSLLKQAGDVEENAVP |
| | | QTAPVPM PDLKNVKSKI GSTENLKHQP GGGKVQIINK |
| | | KLDLSNVQSK CGSKDNIKHV PGGGSVQIVY |
| | | KPVDLSKVTS KCGSLGNIHH KPGGGQVEVK |
| | | SEKLDFKDRV QSKIGSLDNI THVPGGGNKK IE |
| | | DYKDDDDK |
| 17 | Linker | GSGATNFSLLKQAGDVEENAVP |
| 18 | Linker | QQQQS |
| 19 | Linker | QQQQSQQQQSQQQQSQQQQSQQQQS |
| 20 | FLAG epitope | DYKDDDDK |
| 21 | His epitope tag | HHHHHH |
| 22 | HA Epitope Tag | YPYDVPDYA |
| 23 | Mc Epitope | EQKLISEEDL |
| | Tag | |
| 24 | V5 Epitope Tag | GKPIPNPLLGLDST |

### REFERENCES

1. Braak, H., and Braak, E. (1991) Acta Neuropathol 82(4), 239-259
2. Poorkaj, P., Bird, T. D., Wijsman, E., Nemens, E., Garruto, R. M., Anderson, L., Andreadis, A., Wiederholt, W. C., Raskind, M., and Schellenberg, G. D. (1998) Ann Neurol 43(6), 815-825
3. Hutton, M., Lendon, C. L., Rizzu, P., Baker, M., Froelich, S., Houlden, H., Pickering-Brown, S., Chakraverty, S., Isaacs, A., Grover, A., Hackett, J., Adamson, J., Lincoln, S., Dickson, D., Davies, P., Petersen, R. C., Stevens, M., de Graaff, E., Wauters, E., van Baren, J., Hillebrand, M., Joosse, M., Kwon, J. M., Nowotny, P., Che, L. K., Norton, J., Morris, J. C., Reed, L. A., Trojanowski, J., Basun, H., Lannfelt, L., Neystat, M., Fahn, S., Dark, F., Tannenberg, T., Dodd, P. R., Hayward, N., Kwok, J. B., Schofield, P. R., Andreadis, A., Snowden, J., Craufurd, D., Neary, D., Owen, F., Oostra, B. A., Hardy, J., Goate, A., van Swieten, J., Mann, D., Lynch, T., and Heutink, P. (1998) Nature 393(6686), 702-705
4. Pickering-Brown, S., Baker, M., Yen, S. H., Liu, W. K., Hasegawa, M., Cairns, N., Lantos, P. L., Rossor, M., Iwatsubo, T., Davies, Y., Allsop, D., Furlong, R., Owen, F., Hardy, J., Mann, D., and Hutton, M. (2000) Ann Neurol 48(6), 859-867
5. Nacharaju, P., Lewis, J., Easson, C., Yen, S., Hackett, J., Hutton, M., and Yen, S. H. (1999) FEBS Lett 447(2-3), 195-199
6. Spillantini, M. G., Murrell, J. R., Goedert, M., Farlow, M. R., Klug, A., and Ghetti, B. (1998) Proc Natl Acad Sci USA 95(13), 7737-7741
7. Sahara, N., Lewis, J., DeTure, M., McGowan, E., Dickson, D. W., Hutton, M., and Yen, S. H. (2002) J Neurochem 83(6), 1498-1508
8. Ramsden, M., Kotilinek, L., Forster, C., Paulson, J., McGowan, E., SantaCruz, K., Guimaraes, A., Yue, M., Lewis, J., Carlson, G., Hutton, M., and Ashe, K. H. (2005) J Neurosci 25(46), 10637-10647
9. Yoshiyama, Y., Higuchi, M., Zhang, B., Huang, S. M., Iwata, N., Saido, T. C., Maeda, J., Suhara, T., Trojanowski, J. Q., and Lee, V. M. (2007) Neuron 53(3), 337-351
10. Lasagna-Reeves, C. A., Castillo-Carranza, D. L., Sengupta, U., Clos, A. L., Jackson, G. R., and Kayed, R. Mol Neurodegener 6, 39
11. Castillo-Carranza, D. L., Gerson, J. E., Sengupta, U., Guerrero-Munoz, M. J., Lasagna-Reeves, C. A., and Kayed, R. J Alzheimers Dis 40 Suppl 1, S97-S111
12. Peeraer, E., Bottelbergs, A., Van Kolen, K., Stancu, I. C., Vasconcelos, B., Mahieu, M., Duytschaever, H., Ver Donck, L., Torremans, A., Sluydts, E., Van Acker, N., Kemp, J. A., Mercken, M., Brunden, K. R., Trojanowski, J. Q., Dewachter, I., Lee, V. M., and Moechars, D. Neurobiol Dis 73, 83-95
13. Santacruz, K., Lewis, J., Spires, T., Paulson, J., Kotilinek, L., Ingelsson, M., Guimaraes, A., DeTure, M., Ramsden, M., McGowan, E., Forster, C., Yue, M., Ome, J., Janus, C., Mariash, A., Kuskowski, M., Hyman, B., Hutton, M., and Ashe, K. H. (2005) Science 309(5733), 476-481
14. Brunden, K. R., Trojanowski, J. Q., and Lee, V. M. (2008) J Alzheimers Dis 14(4), 393-399
15. Marx, J. (2007) Science 316(5830), 1416-1417
16. Andorfer, C., Acker, C. M., Kress, Y., Hof, P. R., Duff, K., and Davies, P. (2005) J Neurosci 25(22), 5446-5454
17. Polydoro, M., Acker, C. M., Duff, K., Castillo, P. E., and Davies, P. (2009) J Neurosci 29(34), 10741-10749
18. Gomez-Isla, T., Hollister, R., West, H., Mui, S., Growdon, J. H., Petersen, R. C., Parisi, J. E., and Hyman, B. T. (1997) Ann Neurol 41(1), 17-24
19. Terry, R. D. (2000) J Neural Transm Suppl 59, 91-93
20. Conway, K. A., Lee, S. J., Rochet, J. C., Ding, T. T., Williamson, R. E., and Lansbury, P. T., Jr. (2000) Proc Natl Acad Sci USA 97(2), 571-576
21. Lashuel, H. A., Petre, B. M., Wall, J., Simon, M., Nowak, R. J., Walz, T., and Lansbury, P. T., Jr. (2002) J Mol Biol 322(5), 1089-1102
22. Walsh, D. M., Klyubin, I., Fadeeva, J. V., Cullen, W. K., Anwyl, R., Wolfe, M. S., Rowan, M. J., and Selkoe, D. J. (2002) Nature 416(6880), 535-539
23. Cleary, J. P., Walsh, D. M., Hofmeister, J. J., Shankar, G. M., Kuskowski, M. A., Selkoe, D. J., and Ashe, K. H. (2005) Nat Neurosci 8(1), 79-84
24. Novitskaya, V., Bocharova, O. V., Bronstein, I., and Baskakov, I. V. (2006) J Biol Chem 281(19), 13828-13836
25. Berger, Z., Roder, H., Hanna, A., Carlson, A., Rangachari, V., Yue, M., Wszolek, Z., Ashe, K., Knight, J., Dickson, D., Andorfer, C., Rosenberry, T. L., Lewis, J., Hutton, M., and Janus, C. (2007) J Neurosci 27(14), 3650-3662
26. Patterson, K. R., Remmers, C., Fu, Y., Brooker, S., Kanaan, N. M., Vana, L., Ward, S., Reyes, J. F., Philibert, K., Glucksman, M. J., and Binder, L. I. J Biol Chem 286(26), 23063-23076
27. Lasagna-Reeves, C. A., Castillo-Carranza, D. L., Sengupta, U., Sarmiento, J., Troncoso, J., Jackson, G. R., and Kayed, R. Faseb J 26(5), 1946-1959
28. Montejo de Garcini, E., Serrano, L., and Avila, J. (1986) Biochem Biophys Res Commun 141(2), 790-796
29. Wilson, D. M., and Binder, L. I. (1995) J Biol Chem 270(41), 24306-24314
30. DeTure, M. A., Zhang, E. Y., Bubb, M. R., and Purich, D. L. (1996) J Biol Chem 271(51), 32702-32706
31. Khlistunova, I., Biernat, J., Wang, Y., Pickhardt, M., von Bergen, M., Gazova, Z., Mandelkow, E., and Mandelkow, E. M. (2006) J Biol Chem 281(2), 1205-1214
32. Kfoury, N., Holmes, B. B., Jiang, H., Holtzman, D. M., and Diamond, M. I. J Biol Chem 287(23), 19440-19451
33. Congdon, E. E., Kim, S., Bonchak, J., Songrug, T., Matzavinos, A., and Kuret, J. (2008) J Biol Chem 283(20), 13806-13816
34. Friedhoff, P., von Bergen, M., Mandelkow, E. M., Davies, P., and Mandelkow, E. (1998) Proc Natl Acad Sci USA 95(26), 15712-15717
35. King, M. E., Gamblin, T. C., Kuret, J., and Binder, L. I. (2000) J Neurochem 74(4), 1749-1757
36. Friedhoff, P., Schneider, A., Mandelkow, E. M., and Mandelkow, E. (1998) Biochemistry 37(28), 10223-10230
37. Wille, H., Drewes, G., Biernat, J., Mandelkow, E. M., and Mandelkow, E. (1992) J Cell Biol 118(3), 573-584
38. LeVine, H., 3rd. (2004) Anal Biochem 335(1), 81-90
39. Osborn, M. J., Panoskaltsis-Mortari, A., McElmurry, R. T., Bell, S. K., Vignali, D. A., Ryan, M. D., Wilber, A. C., McIvor, R. S., Tolar, J., and Blazar, B. R. (2005) Mol Ther 12(3), 569-574
40. Szymczak, A. L., and Vignali, D. A. (2005) Expert Opin Biol Ther 5(5), 627-638
41. Kim, J. H., Lee, S. R., Li, L. H., Park, H. J., Park, J. H., Lee, K. Y., Kim, M. K., Shin, B. A., and Choi, S. Y. PLoS One 6(4), e18556
48. Sanders, D. W., Kaufman, S. K., DeVos, S. L., Sharma, A. M., Mirbaha, H., Li, A., Barker, S. J., Foley, A. C., Thorpe, J. R., Serpell, L. C., Miller, T. M., Grinberg, L. T., Seeley, W. W., and Diamond, M. I. Neuron 82(6), 1271-1288
49. Frost, B., Jacks, R. L., and Diamond, M. I. (2009) J Biol Chem 284(19), 12845-12852
50. Guo, J. L., and Lee, V. M. J Biol Chem 286(17), 15317-15331
51. Falcon, B., Cavallini, A., Angers, R., Glover, S., Murray, T. K., Barnham, L., Jackson, S., O'Neill, M. J., Isaacs, A. M., Hutton, M. L., Szekeres, P. G., Goedert, M., and Bose, S. J Biol Chem 290(2), 1049-1065
52. Nonaka, T., Watanabe, S. T., Iwatsubo, T., and Hasegawa, M. J Biol Chem 285(45), 34885-34898
57. Abraha, A., Ghoshal, N., Gamblin, T. C., Cryns, V., Berry, R. W., Kuret, J., and Binder, L. I. (2000) J Cell Sci 113 Pt 21, 3737-3745
58. Filipcik, P., Cente, M., Krajciova, G., Vanicky, I., and Novak, M. (2009) Cell Mol Neurobiol 29(6-7), 895-900
59. Zilka, N., Kovacech, B., Barath, P., Kontsekova, E., and Novak, M. Biochem Soc Trans 40(4), 681-686
60. Mena, R., Edwards, P. C., Harrington, C. R., Mukaetova-Ladinska, E. B., and Wischik, C. M. (1996) Acta Neuropathol 91(6), 633-641
61. Chun, W., and Johnson, G. V. (2007) Front Biosci 12, 733-756
62. Binder, L. I., Guillozet-Bongaarts, A. L., Garcia-Sierra, F., and Berry, R. W. (2005) Biochim Biophys Acta 1739(2-3), 216-223
63. Harrington, C. R., Storey, J. M., Clunas, S., Harrington, K. A., Horsley, D., Ishaq, A., Kemp, S. J., Larch, C. P., Marshall, C., Nicoll, S. L., Rickard, J. E., Simpson, M., Sinclair, J. P., Storey, L. J., and Wischik, C. M. J Biol Chem 290(17), 10862-10875
64. Shammas, S. L., Garcia, G. A., Kumar, S., Kjaergaard, M., Horrocks, M. H., Shivji, N., Mandelkow, E., Knowles, T. P., Mandelkow, E., and Klenerman, D. Nat Commun 6, 7025
65. Gamblin, T. C., Berry, R. W., and Binder, L. I. (2003) Biochemistry 42(7), 2252-2257
66. Brandt, R., and Lee, G. (1993) J Biol Chem 268(5), 3414-3419
67. Lee, G., Cowan, N., and Kirschner, M. (1988) Science 239(4837), 285-288
68. Jeganathan, S., von Bergen, M., Brutlach, H., Steinhoff, H. J., and Mandelkow, E. (2006) Biochemistry 45(7), 2283-2293
69. Rosenberg, K. J., Ross, J. L., Feinstein, H. E., Feinstein, S. C., and Israelachvili, J. (2008) Proc Natl Acad Sci USA 105(21), 7445-7450

What is claimed:

1. A tau tandem repeat polypeptide comprising a structure selected from the group consisting of: X₁-L-X₂-T, X₂-L-X₁-T, X₁-X₂-T and X₂-X₁-T wherein:
X₁=Tau
L=linker and
X₂=Tau or a fragment thereof containing the K18 repeat domain, and
T=detectable tag.

2. The tau tandem repeat polypeptide of claim 1, wherein X1=SEQ ID NO: 1, SEQ ID NO:2, SEQ ID NO:3, SEQ ID NO:4, SEQ ID NO:5 or SEQ ID NO:6.

3. The tau tandem repeat polypeptide of claim 1, wherein X2=SEQ ID NO: 1, SEQ ID NO:2, SEQ ID NO:3, SEQ ID NO:4, SEQ ID NO:5, SEQ ID NO:6 or SEQ ID NO:7.

4. The tau tandem repeat polypeptide of claim 1, wherein the T=SEQ ID NO:20.

5. The tau tandem repeat polypeptide of claim 1, wherein L=SEQ ID NO:17, SEQ ID NO:18 or SEQ ID NO:19.

6. The tau tandem repeat polypeptide of claim 1, comprising the amino acid sequence of any one of SEQ ID NOs:11-16.

7. A DNA construct encoding the polypeptide of any one of claim 1.

8. A host cell expressing the DNA construct of claim 7.

9. A non-human transgenic mammal expressing the polypeptide of claim 1.

10. The non-human transgenic mammal of claim 9, wherein the mammal is a mouse.

11. A method of screening for inhibitors of tau aggregation comprising:
i. contacting a test compound with a cell expressing a tau tandem repeat polypeptide according to claim 1, for a time sufficient to allow the formation of tau tandem repeat oligomers,
ii. measuring the presence of tau tandem repeat oligomers using an assay that is able to differentiate between tau tandem repeats monomers and tau tandem repeat oligomers;
wherein a decrease in the presence of tau oligomers relative to control indicates that the compound is an inhibitor of tau aggregation.

12. The method of claim 11, wherein the measuring step uses a bead based immunoassay comprising an acceptor bead linked to a first antibody and a donor bead linked to a second antibody.

13. The method of claim 12, wherein the first and the second antibody specifically bind to the detectable tag (T).
